SEQUENCE LISTING (1 sequence)

SEQ ID NO: 1 - DNA, 24 bp, Artificial
  Synthesized
gacgtcgacgttaacgtcaacgtt

(19) United States
(12) Patent Application Publication
(10) Pub. No.: US 2006/0120997 A1
(43) Pub. Date: Jun. 8, 2006

(54) Cancer therapeutic compositions

(75) Inventor: James S. Lipton, West Palm Beach, FL (US)

Correspondence Address: AKERMAN SENTERFITT, P.O. BOX 3188, WEST PALM BEACH, FL, 33402-3188, US

(73) Assignee: BIOMUNE, INC., Palm Beach, FL (US)

(21) Appl. No.: 11/263,581

(22) Filed: Oct. 31, 2005

Related U.S. Application Data
(60) Provisional application No. 60/623,514, filed on Oct. 29, 2004.

(51) Int. Cl. A61K 38/20; A61K 39/00; A61K 36/185

(52) U.S. Cl. 424/85.2; 424/185.1; 424/725

(57) ABSTRACT

A pharmaceutical composition comprising lectins is anti-tumorigenic. The composition is used in imaging, diagnosis and therapy of cancer.

### CROSS-REFERENCE TO RELATED APPLICATIONS

This application claims the benefit of U.S. Provisional Application No. 60/623,514, filed Oct. 29, 2004, which is incorporated herein by reference in its entirety.

### FIELD OF THE INVENTION

The invention relates to compositions and methods for targeting and modulating the activity of tumor cells and cells infected by cancer causing infectious disease agents. In particular, the invention relates to inhibition of immunoglobulin isoclass switching.

### BACKGROUND

Cancer is one of the leading causes of disease, being responsible for 526,000 deaths in the United States each year. For example, breast cancer is the most common form of malignant disease among women in Western countries and, in the United States, is the most common cause of death among women between 40 and 55 years of age (Forrest, 1990). The incidence of breast cancer is increasing, especially in older women, but the cause of this increase is unknown. Malignant melanoma is another form of cancer whose incidence is increasing at a frightening rate, at least sixfold in the United States since 1945, and is the single most deadly of all skin diseases.

One of the most devastating aspects of cancer is the propensity of cells from malignant neoplasms to disseminate from their primary site to distant organs and develop into metastases. Despite advances in surgical treatment of primary neoplasms and aggressive therapies, most cancer patients die as a result of metastatic disease. Animal tests indicate that about 0.01% of circulating cancer cells from solid tumors establish successful metastatic colonies.

Thus there is a need in the art to establish agents for therapy of cancer without the side effects observed with conventional therapies.

### SUMMARY

Compositions for the treatment of cancer decrease IgG production and stimulate IgM production. In particular, the compositions bind to tumor antigens producing an IgM dominant response. The binding of the compositions also allows for imaging of a tumor.

In a preferred embodiment, a composition comprising lectins is used to image, diagnose and treat cancers. A source of lectins includes, but is not limited to plants such as soybean, wheatgerm, mistletoe, and pokeweed. Preferably, these lectins are sensitive to the N-acetyl glucosamine epitope.

In a preferred embodiment, the compositions of the invention comprise, as active ingredients, pokeweed mitogen (PWN), and poly(I:C) in a 1:1 ratio v/v. The composition can also comprise phytohemmagglutinin (PHA). Preferably, the PWN-PHA-poly IC compositions are in a 1:0.5:1 ratio respectively and the PWM is modified by PEG. The Poly(I:C) is enclosed in a cationic liposome of 1:1 molar ratio of DOTAP-cholesterol. DOTAP=1,2 dioleol-3-trimethylammonium-propoane. Lipid/RNA ratio set at 32 μmol lipid to 1.0 microgram RNA, or equivalents thereof, preferably

In another preferred embodiment, the pharmaceutical composition further comprises recombinant Interleukin 2 (rIL-2) of about 100,000 U/kg.

In another preferred embodiment, a method of treating a cancer patient, comprises administering to a patient in need thereof, a composition comprising pokeweed mitogen; phytohemmaglutinin; and, poly(I:C); contacting a tumor cell with the composition; thereby treating the cancer patient. Preferably the composition comprises about 1% up to 99% w/w of pokeweed mitogen; phytohemmaglutinin; and, poly(I:C) in a ratio of 1:0.5:1 v/v respectively, more preferably, the composition comprises about 1% to about 20% w/w of pokeweed mitogen; phytohemmaglutinin; and, poly(I:C) in a ratio of 1:0.5:1 v/v respectively.

In one aspect of the invention, the composition comprising the pokeweed mitogen; phytohemmaglutinin; and, poly(I:C) in a ratio of 1:0.5:1 v/v respectively is formulated as a topical cream and can be administered with one or more chemotherapeutic agents. Preferably, the chemotherapeutic agent is co-administered, precede, or administered after the composition comprising a therapeutic effective amount of the pokeweed mitogen, phytohemmaglutinin, poly(I:C) composition.

In another preferred embodiment, a method of tumor imaging using X-ray, single-photon-emission tomography(SPECT), magnetic resonance imaging (MRI), or positron emission tomography (PET), comprising administering a pharmaceutically acceptable composition containing detectably labeled PWM-poly IC-, or a detectably labeled PWM molecule or an anti-PWM antibody.

In one preferred embodiment, the detectable label is selected from the group consisting of: copper⁶⁷, gallium⁶⁷, gallium⁶⁸, indium¹¹¹, indium¹¹³, iodine¹²³, iodine¹²⁵, iodine¹³¹, mercury¹⁹⁷, mercury²⁰³, rhenium¹⁸⁶, rhenium¹⁸⁸, rubidium⁹⁷, rubidium¹⁰³, technetium^{99m} and yttrium⁹⁰.

In another preferred embodiment, the detectable label is also selected from the group consisting of: cobalt (II), copper (II), chromium (III), dysprosium (III), erbium (III), gadolinium (III), holmium (III), iron (II), iron (III), manganese (II), neodymium (III), nickel (II), samarium (III), terbium (III), vanadium (II), ytterbium (III); rhodamine or fluorescein.

In yet another preferred embodiment, the detectable label is selected from the group consisting of: chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), or erbium (III).

In a preferred embodiment, a kit comprises pokeweed mitogen; phytohemmaglutinin; and, poly(I:C).

In another preferred embodiment, isolated lectins stimulate B-lymphocytes and produces antibodies. Preferably the lectin is pokeweed mitogen (PWM). Not only is PWM a mitogen for B cells, it is an antigen that stimulates anti-PWM antibodies.

In another preferred embodiment, the compositions inhibit isoclass immunoglobulin switching. Inhibition of isoclass switching is important for the fixing of complement of targeted cells, i.e. those coated with immunoglobulin, preferably, IgM.

In another preferred embodiment, the PWM is modified by PEG.

In a preferred embodiment, PWM delivery is engineered to target the tumor antigen wherein the pokeweed mitogen binds to the tumor antigen and B cells produce antibodies specific for the tumor antigen to which PWM has bound to.

In a preferred embodiment, the anti-cancer, PWM-poly IC or PWM-PHA-poly IC compositions are highly cytotoxic for tumors, specific for tumor antigens only and, universal for all tumor antigens.

In another preferred embodiment, the anti-cancer composition comprises pokeweed mitogen which can be modified by polyethylene glycol as described in the Examples which follow.

In another preferred embodiment, the anti-cancer composition comprises IgM. IgM antibodies are capable of destroying a ‘foreign’ cell. Since there are multiple tumor antigens, all with differing protein structure, an antibody specific for one would necessarily be wrong for the others but what conventional wisdom misses is that tumor antigens are not just proteins; they are glycoproteins.

In another preferred embodiment, the PWM-poly IC or PWM-PHA-poly IC compositions comprising lectins such as helix pomatia can be used in conjunction with chemotherapeutic agents. The composition can be administered to a patient in combination with metronomic therapy. For example, administration of continuous low-doses of the chimeric fusion molecule and one or more therapeutic agents. Therapeutic agents can include, for example, chemotherapeutic agents such as, cyclophosphamide (CTX 25 mg/kg/day, p.o.), taxanes (paclitaxel or docetaxel), busulfan, cisplatin, cyclophosphamide, methotrexate, daunorubicin, doxorubicin, melphalan, cladribine, vincristine, vinblastine, and chlorambucil.

Other aspects of the invention are described infra.

### DETAILED DESCRIPTION

The invention provides compositions and methods for diagnosing, imaging and treating cancer. In particular, the compositions are found to inhibit isoclass switching from IgM to IgG. The below described preferred embodiments illustrate adaptations of these compositions and methods. Nonetheless, from the description of these embodiments, other aspects of the invention can be made and/or practiced based on the description provided below.

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

### DEFINITIONS

In accordance with the present invention and as used herein, the following terms are defined with the following meanings, unless explicitly stated otherwise.

As used herein, “a”, “an,” and “the” include plural references unless the context clearly dictates otherwise.

“Label molecules” are chemical or biochemical moieties used for labeling a compound such as, for example, pokeweed, polynucleotide, polypeptide, or antibody. They include, but are not limited to, radionuclides, enzymes, substrates, cofactors, inhibitors, fluorescent agents, chromogenic agents, chemiluminescent agents, magnetic particles, and the like. Reporter molecules specifically bind, establish the presence of, and allow quantification of a particular polynucleotide, polypeptide, or antibody.

“Sample” is used herein in its broadest sense. A sample suspected of containing a nucleic acid can comprise a cell, chromosomes isolated from a cell (e.g., a spread of metaphase chromosomes), genomic DNA, RNA, cDNA and the like. A sample comprising polynucleotides, polypeptides, peptides, antibodies and the like may comprise a bodily fluid; a soluble fraction of a cell preparation, or media in which cells were grown; a chromosome, an organelle, or membrane isolated or extracted from a cell; genomic DNA, RNA, or cDNA, polypeptides, or peptides in solution or bound to a substrate; a cell; a tissue; a tissue print; a fingerprint, skin or hair; and the like.

“Substantially purified” refers to nucleic acid molecules or proteins that are removed from their natural environment and are isolated or separated, and are at least about 60% free, preferably about 75% free, and most preferably about 90% free, from other components with which they are naturally associated.

As used herein, “cancer” refers to all types of cancer or neoplasm or malignant tumors found in mammals, including, but not limited to: leukemias, lymphomas, melanomas, carcinomas and sarcomas. Examples of cancers are cancer of the brain, breast, pancreas, cervix, colon, head & neck, kidney, lung, non-small cell lung, melanoma, mesothelioma, ovary, sarcoma, stomach, uterus and Medulloblastoma.

Additional cancers which can be treated by the disclosed composition according to the invention include but not limited to, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, urinary bladder cancer, premalignant skin lesions, testicular cancer, lymphomas, thyroid cancer, neuroblastoma, esophageal cancer, genitourinary tract cancer, malignant hypercalcemia, cervical cancer, endometrial cancer, adrenal cortical cancer, and prostate cancer.

The term “leukemia” refers broadly to progressive, malignant diseases of the blood-forming organs and is generally characterized by a distorted proliferation and development of leukocytes and their precursors in the blood and bone marrow. Leukemia is generally clinically classified on the basis of (1) the duration and character of the disease-acute or chronic; (2) the type of cell involved; myeloid (myelogenous), lymphoid (lymphogenous), or monocytic; and (3) the increase or non-increase in the number of abnormal cells in the blood-leukemic or aleukemic (subleukemic). Accordingly, the present invention includes a method of treating leukemia, and, preferably, a method of treating acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

The term “sarcoma” generally refers to a tumor which is made up of a substance like the embryonic connective tissue and is generally composed of closely packed cells embedded in a fibrillar or homogeneous substance. Examples of sarcomas which can be treated with the compositions and optionally a potentiator and/or chemotherapeutic agent include, but not limited to a chondrosarcoma, fibrosarcoma, lymphosarcoma, melanosarcoma, myxosarcoma, osteosarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

The term “melanoma” is taken to mean a tumor arising from the melanocytic system of the skin and other organs. Melanomas which can be treated with the compositions and optionally a potentiator and/or another chemotherapeutic agent include but not limited to, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

The term “carcinoma” refers to a malignant new growth made up of epithelial cells tending to infiltrate the surrounding tissues and give rise to metastases. Carcinomas which can be treated with the compositions and optionally a potentiator and/or a chemotherapeutic agent include but not limited to, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriforn carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale adenoides, exophytic carcinoma, carcinoma ex ulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, carcinoma scroti, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrucous carcinoma, and carcinoma villosum.

As used herein, “variant” of polypeptides refers to an amino acid sequence that is altered by one or more amino acid residues. The variant may have “conservative” changes, wherein a substituted amino acid has similar structural or chemical properties (e.g., replacement of leucine with isoleucine). More rarely, a variant may have “nonconservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, LASERGENE software (DNASTAR).

The term “variant,” when used in the context of a polynucleotide sequence, may encompass a polynucleotide sequence related to a wild type gene. This definition may also include, for example, “allelic”, “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type target gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

The resulting polypeptides generally will have significant amino acid identity relative to each other. A polymorphic variant is a variation in the polynucleotide sequence of a particular gene between individuals of a given species. Polymorphic variants also may encompass “single nucleotide polymorphisms” (SNPs,) or single base mutations in which the polynucleotide sequence varies by one base. The presence of SNPs may be indicative of, for example, a certain population with a propensity for a disease state, that is susceptibility versus resistance.

“Diagnostic” or “diagnosed” means identifying the presence or nature of a pathologic condition or a patient susceptible to a disease. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, a “pharmaceutically acceptable” component is one that is suitable for use with humans and/or animals without undue adverse side effects (such as toxicity, irritation, and allergic response) commensurate with a reasonable benefit/risk ratio.

As used herein, the term “safe and effective amount” refers to the quantity of a component which is sufficient to yield a desired therapeutic response without undue adverse side effects (such as toxicity, irritation, or allergic response) commensurate with a reasonable benefit/risk ratio when used in the manner of this invention. By “therapeutically effective amount” is meant an amount of a compound of the present invention effective to yield the desired therapeutic response. For example, an amount effective to delay the growth of or to cause a cancer, either a sarcoma or lymphoma, or to shrink the cancer or prevent metastasis. The specific safe and effective amount or therapeutically effective amount will vary with such factors as the particular condition being treated, the physical condition of the patient, the type of mammal or animal being treated, the duration of the treatment, the nature of concurrent therapy (if any), and the specific formulations employed and the structure of the compounds or its derivatives.

As used herein, a “pharmaceutical salt” include, but are not limited to, mineral or organic acid salts of basic residues such as amines; alkali or organic salts of acidic residues such as carboxylic acids. Preferably the salts are made using an organic or inorganic acid. These preferred acid salts are chlorides, bromides, sulfates, nitrates, phosphates, sulfonates, formates, tartrates, maleates, malates, citrates, benzoates, salicylates, ascorbates, and the like. The most preferred salt is the hydrochloride salt.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

The treatment of neoplastic disease or neoplastic cells, refers to an amount of the composition, vectors and/or peptides, described throughout the specification and in the Examples which follow, capable of invoking one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention of tumor cell infiltration into peripheral organs; (6) inhibition, including (i) reduction, (ii) slowing down or (iii) complete prevention of metastasis; (7) enhancement of anti-tumor immune response, which may result in (i) maintaining tumor size, (ii) reducing tumor size, (iii) slowing the growth of a tumor, (iv) reducing, slowing or preventing invasion or (v) reducing, slowing or preventing metastasis; and/or (8) relief, to some extent, of one or more symptoms associated with the disorder.

Treatment of an individual suffering from an infectious disease organism refers to a decrease and elimination of the disease organism from an individual. For example, a decrease of viral particles as measured by plaque forming units or other automated diagnostic methods such as ELISA etc.

As used herein “immunogenicity modifier” refers to a decreased immune recognition against an antigen, i.e. a non-immunogenic antigen.

A “chemotherapeutic agent” is a chemical compound useful in the treatment of cancer. Examples of chemotherapeutic agents include alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carnomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g. paclitaxel (TAXOL®, Bristol-Myers Squibb Oncology, Princeton, N.J.) and docetaxel (TAXOTERE®, Rhône-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylomithine (DMFO); retinoic acid; esperamicins; capecitabine; and pharmaceutically acceptable salts, acids or derivatives of any of the above. Also included in this definition are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

By the term “modulate,” it is meant that any of the mentioned activities, are, e.g., increased, enhanced, increased, agonized (acts as an agonist), promoted, decreased, reduced, suppressed blocked, or antagonized (acts as an agonist). Modulation can increase activity more than 1-fold, 2-fold, 3-fold, 5-fold, 10-fold, 100-fold, etc., over baseline values. Modulation can also decrease its activity below baseline values.

“Cells of the immune system” or “immune cells” as used herein, is meant to include any cells of the immune system that may be assayed, including, but not limited to, B lymphocytes, also called B cells, T lymphocytes, also called T cells, natural killer (NK) cells, natural killer T (NK) cells, lymphokine-activated killer (LAK) cells, monocytes, macrophages, neutrophils, granulocytes, mast cells, platelets, Langerhans cells, stem cells, dendritic cells, peripheral blood mononuclear cells, tumor-infiltrating (TIL) cells, gene modified immune cells including hybridomas, drug modified immune cells, and derivatives, precursors or progenitors of the above cell types.

“Immune effector cells” refers to cells capable of binding an antigen and which mediate an immune response selective for the antigen. These cells include, but are not limited to, T cells (T lymphocytes), B cells (B lymphocytes), monocytes, macrophages, natural killer (NK) cells and cytotoxic T lymphocytes (CTLs), for example CTL lines, CTL clones, and CTLs from tumor, inflammatory, or other infiltrates.

“Immune related molecules” refers to any molecule identified in any immune cell, whether in a resting (“non-stimulated”) or activated state, and includes any receptor, ligand, cell surface molecules, nucleic acid molecules, polypeptides, variants and fragments thereof.

A “chemokine” is a small cytokine involved in the migration and activation of cells, including phagocytes and lymphocytes, and plays a role in inflammatory responses.

A “cytokine” is a protein made by a cell that affect the behavior of other cells through a “cytokine receptor” on the surface of the cells the cytokine effects. Cytokines manufactured by lymphocytes are sometimes termed “lymphokines.” Cytokines are also characterized as Type I (e.g. IL-2 and IFN-β) and Type II (e.g. IL-4 and IL-10).

An “epitope”, as used herein, is a portion of a polypeptide that is recognized (i.e., specifically bound) by a B-cell and/or T-cell surface antigen receptor. Epitopes may generally be identified using well known techniques, such as those summarized in Paul, Fundamental Immunology, 3rd ed., 243-247 (Raven Press, 1993) and references cited therein. Such techniques include screening polypeptides derived from the native polypeptide for the ability to react with antigen-specific antisera and/or T-cell lines or clones. An epitope of a polypeptide is a portion that reacts with such antisera and/or T-cells at a level that is similar to the reactivity of the full length polypeptide (e.g., in an ELISA and/or T-cell reactivity assay). Such screens may generally be performed using methods well known to those of ordinary skill in the art, such as those described in Harlow and Lane, Antibodies: A Laboratory Manual, Cold Spring Harbor Laboratory, 1988. B-cell and T-cell epitopes may also be predicted via computer analysis.

“Immunoassay” is an assay that uses an antibody to specifically bind an antigen (e.g., a marker). The immunoassay is characterized by the use of specific binding properties of a particular antibody to isolate, target, and/or quantify the antigen.

“Activity”, “activation” or “augmentation” is the ability of immune cells to respond and exhibit, on a measurable level, an immune function. Measuring the degree of activation refers to a quantitative assessment of the capacity of immune cells to express enhanced activity when further stimulated as a result of prior activation. The enhanced capacity may result from biochemical changes occurring during the activation process that allow the immune cells to be stimulated to activity in response to low doses of stimulants.

“Immune cell activity” as used herein refers to the activation of any immune cell. Activity that may be measured include, but is not limited to, (1) cell proliferation by measuring the DNA replication; (2) enhanced cytokine production, including specific measurements for cytokines, such as IFN-γ, GM-CSF, or TNF-α (3) cell mediated target killing or lysis; (4) cell differentiation; (5) immunoglobulin production; (6) phenotypic changes; (7) production of chemotactic factors or chemotaxis, meaning the ability to respond to a chemotactin with chemotaxis; (8) immunosuppression, by inhibition of the activity of some other immune cell type; and, (9) apoptosis, which refers to fragmentation of activated immune cells under certain circumstances, as an indication of abnormal activation.

Compositions

In a preferred embodiment, the compositions of the invention comprise, as active ingredients, pokeweed mitogen (PWN), and poly(I:C) in a 1:1 ratio v/v. The composition can also comprise phytohemmagglutinin (PHA). Preferably, the PWN-PHA-poly IC compositions are in a 1:0.5:1 ratio respectively. The Poly(I:C) is enclosed in a cationic liposome of 1:1 molar ratio of DOTAP-cholesterol. DOTAP=1,2 dioleol-3-trimethylammonium-propoane. Lipid/RNA ratio set at 32 nmol lipid to 1.0 microgram RNA, or equivalents thereof.

In another preferred embodiment, the composition inhibits isoclass switching and results in the lysis of tumor cells and/or cells infected with an infectious disease organism. Without wishing to be bound by theory, antigen specific IgM detects abnormal antigens, fixes complement with a higher efficiency than IgG and initiates the complement cascade thereby resulting in the lysis of such cells.

Several polynucleotides have been extensively evaluated as biological response modifiers. Perhaps the best example is poly (I,C) which is a potent inducer of IFN production as well as macrophage activator and inducer of NK activity (Talmadge, J. E., J. Adams, H. Phillips, M. Collins, B. Lenz, M. Schneider, E. Schlick, R. Ruffinann, R. H. Wiltrout, and M. A. Chirigos. 1985. “Immunomodulatory effects in mice of polyinosinic-polycytidylic acid complexed with poly-L-lysine and carboxymethylcellulose.” Cancer Res. 45:1058; Wiltrout, R. H., R. R. Salup, T. A. Twilley, and J. E. Talmadge. 1985. “Immunomodulation of natural killer activity by polyribonucleotides.” J. Biol. Respn. Mod. 4:512; Krown, S. E. 1986. “Interferons and interferon inducers in cancer treatment.” Sem. Oncol. 13:207; and Ewel, C. H., S. J. Urba, W. C. Kopp, J. W. Smith II, R. G. Steis, J. L. Rossio, D. L. Longo, M. J. Jones, W. G. Alvord, C. M. Pinsky, J. M. Beveridge, K. L. McNitt, and S. P. Creekmore. 1992. “Polyinosinic-polycytidylic acid complexed with poly-L-lysine and carboxymethylcellulose in combination with interleukin-2 in patients with cancer: clinical and immunological effects.” Canc. Res. 52:3005).

In a preferred embodiment, a composition comprising lectins is used to image, diagnose and treat cancers. A source of lectins includes, but is not limited to plants such as soybean, wheatgerm, mistletoe, and pokeweed. Preferably, these lectins are sensitive to the N-acetyl glucosamine epitope. In collagen, a prevalent glycoprotein in the extra-cellular matrix, the terminal oligosaccharides are linked to oxygen i.e., O-linked. Collagen is “too sticky” to allow cancer cells to separate and metastasize. In contrast, chitin, the most abundant glycoprotein in the insect world is not “sticky” at all; it is smooth and non-adherent. The terminal sugars in chitin are linked to nitrogen, N-linked. Without wishing to be bound by theory, the N-linked glycoprotein extra-cellular matrix might be more amenable to metastasis than an O-linked matrix.

In another preferred embodiment, isolated lectins stimulate B-lymphocytes and produces antibodies. Preferably the lectin is pokeweed mitogen (PWM). Not only is PWM a mitogen for B cells, it is an antigen that stimulates anti-PWM antibodies. .

In a preferred embodiment, PWM delivery is engineered to target the tumor antigen, then produce an antibody ‘bullet’ to shoot at this target.

In a preferred embodiment, the anti-cancer compositions are highly cytotoxic for tumors, specific for tumor antigens only and, universal for all tumor antigens.

In another preferred embodiment, the anti-cancer composition comprises pokeweed mitogen.

In another preferred embodiment, the anti-cancer composition comprises IgM. IgM antibodies are capable of destroying a ‘foreign’ cell. Since there are multiple tumor antigens, all with differing protein structure, an antibody specific for one would necessarily be wrong for the others but what conventional wisdom misses is that tumor antigens are not just proteins; they are glycoproteins.

In another preferred embodiment, the compositions minimize the amount of IgG formed. Unmodified PWM produces IgG and IgM in roughly equivalent amounts. Modification with polyethylene glycol or dinitrophenol decreases IgG production. We were able to substantially decrease IgG production by coating 50% of the molecule with PEG.

While it is possible for the composition to be administered alone, it is preferable to present it as a pharmaceutical formulation. The active ingredient may comprise, for topical administration, from 0.001% to 10% w/w, e.g., from 1% to 2% by weight of the formulation, although it may comprise as much as 10% w/w but preferably not in excess of 5% w/w and more preferably from 0.1% to 1% w/w of the formulation. The topical formulations of the present invention, comprise an active ingredient together with one or more acceptable carrier(s) therefor and optionally any other therapeutic ingredients(s). The carrier(s) must be “acceptable” in the sense of being compatible with the other ingredients of the formulation and not deleterious to the recipient thereof.

Formulations suitable for topical administration include liquid or semi-liquid preparations suitable for penetration through the skin to the site of where treatment is required, such as liniments, lotions, creams; ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

The composition of the invention can be administered to a patient either by themselves, or in pharmaceutical compositions where it is mixed with suitable carriers or excipient(s). In treating a patient exhibiting a disorder of interest, a therapeutically effective amount of a agent or agents such as these is administered. A therapeutically effective dose refers to that amount of the compound that results in amelioration of symptoms or a prolongation of survival in a patient.

Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds which exhibit large therapeutic indices are preferred. The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized.

For any compound used in the method of the invention, the therapeutically effective dose can be estimated initially from cell culture assays. For example, a dose can be formulated in animal models to achieve a circulating plasma concentration range that includes the IC₅₀ as determined in cell culture. Such information can be used to more accurately determine useful doses in humans. Levels in plasma may be measured, for example, by HPLC.

The exact formulation, route of administration and dosage can be chosen by the individual physician in view of the patient's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions. Conversely, the attending physician would also know to adjust treatment to higher levels if the clinical response were not adequate (precluding toxicity). The magnitude of an administrated dose in the management of the oncogenic disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient. A program comparable to that discussed above may be used in veterinary medicine.

Depending on the specific conditions being treated, such agents may be formulated and administered systemically or locally. Techniques for formulation and administration may be found in Remington's Pharmaceutical Sciences, 18^{th} ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

The compositions described above may be administered to a subject in any suitable formulation. In addition to treatment of cancer with topical formulations of the composition, in other aspects of the invention the composition can be delivered by other methods. For example, the composition can be formulated for parenteral delivery, e.g., for subcutaneous, intravenous, intramuscular, or intratumoral injection. Other methods of delivery, for example, liposomal delivery or diffusion from a device impregnated with the composition might be used. The compositions may be administered in a single bolus, multiple injections, or by continuous infusion (for example, intravenously or by peritoneal dialysis). For parenteral administration, the compositions are preferably formulated in a sterilized pyrogen-free form. Compositions of the invention can also be administered in vitro to a cell (for example, to induce apoptosis in a cancer cell in an in vitro culture) by simply adding the composition to the fluid in which the cell is contained.

Depending on the specific conditions being treated, such agents may be formulated and administered systemically or locally. Techniques for formulation and administration may be found in Remington's Pharmaceutical Sciences, 18^{th} ed., Mack Publishing Co., Easton, Pa. (1990). Suitable routes may include oral, rectal, transdermal, vaginal, transmucosal, or intestinal administration; parenteral delivery, including intramuscular, subcutaneous, intramedullary injections, as well as intrathecal, direct intraventricular, intravenous, intraperitoneal, intranasal, or intraocular injections, just to name a few.

For injection, the agents of the invention may be formulated in aqueous solutions, preferably in physiologically compatible buffers such as Hanks's solution, Ringer's solution, or physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm. Additionally, due to their hydrophobicity, small organic molecules may be directly administered intracellularly.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Formulations suitable for topical administration include liquid or semi-liquid preparations suitable for penetration through the skin to the site of where treatment is required, such as liniments, lotions, creams, ointments or pastes, and drops suitable for administration to the eye, ear, or nose. Drops according to the present invention may comprise sterile aqueous or oily solutions or suspensions and may be prepared by dissolving the active ingredient in a suitable aqueous solution of a bactericidal and/or fungicidal agent and/or any other suitable preservative, and preferably including a surface active agent. The resulting solution may then be clarified and sterilized by filtration and transferred to the container by an aseptic technique. Examples of bactericidal and fungicidal agents suitable for inclusion in the drops are phenylmercuric nitrate or acetate (0.002%), benzalkonium chloride (0.01%) and chlorhexidine acetate (0.01%). Suitable solvents for the preparation of an oily solution include glycerol, diluted alcohol and propylene glycol.

Lotions according to the present invention include those suitable for application to the skin or eye. An eye lotion may comprise a sterile aqueous solution optionally containing a bactericide and may be prepared by methods similar to those for the preparation of drops. Lotions or liniments for application to the skin may also include an agent to hasten drying and to cool the skin, such as an alcohol or acetone, and/or a moisturizer such as glycerol or an oil such as castor oil or arachis oil.

Creams, ointments or pastes according to the present invention are semi-solid formulations of the active ingredient for external application. They may be made by mixing the active ingredient in finely-divided or powdered form, alone or in solution or suspension in an aqueous or non-aqueous fluid, with the aid of suitable machinery, with a greasy or non-greasy basis. The basis may comprise hydrocarbons such as hard, soft or liquid paraffin, glycerol, beeswax, a metallic soap; a mucilage; an oil of natural origin such as almond, corn, arachis, castor or olive oil; wool fat or its derivatives, or a fatty acid such as stearic or oleic acid together with an alcohol such as propylene glycol or macrogels. The formulation may incorporate any suitable surface active agent such as an anionic, cationic or non-ionic surface active such as sorbitan esters or polyoxyethylene derivatives thereof. Suspending agents such as natural gums, cellulose derivatives or inorganic materials such as silicaceous silicas, and other ingredients such as lanolin, may also be included.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxy-methylcellulose, and/or polyvinyl pyrrolidone (PVP). If desired, disintegrating agents may be added, such as the cross-linked polyvinyl pyrrolidone, agar, or alginic acid or a salt thereof such as sodium alginate.

Dragee cores are provided with suitable coating. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added.

The composition can include a buffer system, if desired. Buffer systems are chosen to maintain or buffer the pH of compositions within a desired range. The term “buffer system” or “buffer” as used herein refers to a solute agent or agents which, when in a water solution, stabilize such solution against a major change in pH (or hydrogen ion concentration or activity) when acids or bases are added thereto. Solute agent or agents which are thus responsible for a resistance or change in pH from a starting buffered pH value in the range indicated above are well known. While there are countless suitable buffers, potassium phosphate monohydrate is a preferred buffer.

The final pH value of the pharmaceutical composition may vary within the physiological compatible range. Necessarily, the final pH value is one not irritating to human skin and preferably such that transdernal transport of the active compound, i.e. PWM-poly IC-PHA is facilitated. Without violating this constraint, the pH may be selected to improve the compound stability and to adjust consistency when required. In one embodiment, the preferred pH value is about 3.0 to about 7.4, more preferably about 3.0 to about 6.5, most preferably from about 3.5 to about 6.0.

For preferred topical delivery vehicles the remaining component of the composition is water, which is necessarily purified, e.g., deionized water. Such delivery vehicle compositions contain water in the range of more than about 50 to about 95 percent, based on the total weight of the composition. The specific amount of water present is not critical, however, being adjustable to obtain the desired viscosity (usually about 50 cps to about 10,000 cps) and/or concentration of the other components. The topical delivery vehicle preferably has a viscosity of at least about 30 centipoises.

Other known transdermal skin penetration enhancers can also be used to facilitate delivery of the PWM-poly IC or PWM-poly IC-PHA composition. Illustrative are sulfoxides such as dimethylsulfoxide (DMSO) and the like; cyclic amides such as 1-dodecylazacycloheptane-2-one (Azone™, a registered trademark of Nelson Research, Inc.) and the like; amides such as N,N-dimethyl acetamide (DMA) N,N-diethyl toluamide, N,N-dimethyl formamide, N,N-dimethyl octamide, N,N-dimethyl decamide, and the like; pyrrolidone derivatives such as N-methyl-2-pyrrolidone, 2-pyrrolidone, 2-pyrrolidone-5-carboxylic acid, N-(2-hydroxyethyl)-2-pyrrolidone or fatty acid esters thereof, 1-lauryl-4-methoxycarbonyl-2-pyrrolidone, N-tallowalkylpyrrolidones, and the like; polyols such as propylene glycol, ethylene glycol, polyethylene glycol, dipropylene glycol, glycerol, hexanetriol, and the like; linear and branched fatty acids such as oleic, linoleic, lauric, valeric, heptanoic, caproic, myristic, isovaleric, neopentanoic, trimethyl hexanoic, isostearic, and the like; alcohols such as ethanol, propanol, butanol, octanol, oleyl, stearyl, linoleyl, and the like; anionic surfactants such as sodium laurate, sodium lauryl sulfate, and the like; cationic surfactants such as benzalkonium chloride, dodecyltrimethylammonium chloride, cetyltrimethylammonium bromide, and the like; non-ionic surfactants such as the propoxylated polyoxyethylene ethers, e.g., Poloxamer 231, Poloxamer 182, Poloxamer 184, and the like, the ethoxylated fatty acids, e.g., Tween 20, Myrj 45, and the like, the sorbitan derivatives, e.g., Tween 40, Tween 60, Tween 80, Span 60, and the like, the ethoxylated alcohols, e.g., polyoxyethylene (4) lauryl ether (Brij 30), polyoxyethylene (2) oleyl ether (Brij 93), and the like, lecithin and lecithin derivatives, and the like; the terpenes such as D-limonene, α-pinene, β-carene, α-terpineol, carvol, carvone, menthone, limonene oxide, α-pinene oxide, eucalyptus oil, and the like. Also suitable as skin penetration enhancers are organic acids and esters such as salicyclic acid, methyl salicylate, citric acid, succinic acid, and the like.

In another preferred embodiment, the composition comprises one or more isolated lectins. The most preferred is pokeweed mitogen. Examples of lectins in addition to lectins isolated from Helix pomatia include but not limited to: Anguilla anguilla (Eel serum); Aleuria aurantia (Orange peel fungus); Agaricus bisporus (Mushroom); Amphicarpanea bracteata (hog-peanut); Hippaestrum hybrid (Amaryllis bulbs); Abrus precatorius (Jequirity bean); Bauhinia purpurea alba (camel's foot tree); Caragana arborescens (Siberian pea tree); Concanavalia ensiformis (Jack bean); Cicer arietinum (chick pea); Cytisus scoparius (Scotch broom); Colichos biflorus (horse gram); Datura stramonium (Jimson weed, Thorn apple); Erythrina crystagalli (Coral tree); Erythrina coralldendron (Coral tree); Euonymus europaeus (spindle tree); Dolichos biflorus (horse gram); Galanthus nivalis (Snowdrop bulb); Griffonia simplicifolia; Helix aspersa (Garden snail); Artocarpus integrifolia (jackfruit); Laburnum alpinum; Phaseolus lunatis (also limensis) (Lima bean); Lens culinaris (lentil); Lycopersicon esculentum (Tomato); Lathyrus oderatus (Sweet pea); Lotus tetragonolobus (Asparagus pea); Maackla amurensis (maackla); Maclura pomifera (Osage orange); Narcissus pseudonarcissus (daffodil); Phytolacca americana (Pokeweed); Phaseolis vulgaris (Red kidney bean); Arachis hypogaea (Peanut); Pisum sativum (Pea); Phytolacca americana (pokeweed); Psophocarpus tetagonolobus (winged bean); Psophocarpus tetagonolobus (winged bean); Ricinus communis (Castor bean); Robinia pseudoaccacia (black locust); Glycine max (Soybean); Sophora japonica (Japanese pagoda tree); Solanum tuberosum (Potato); Trichosanthes kinlowii (China gourd); Ulex europaeus (Gorse or Furz seeds); Viscum album (European mistletoe); Vicia faba (Fava bean); Vicia graminea; Vigna radiata (mung bean); Vicia sativa; Vicia villosa (Hairy vetch); Wisteria floribunda (Japanese wisteria); Triticum vulgaris (Wheat germ); Succinyl WGA.

In accordance with the invention any one or more, or combinations thereof with pokeweed mitogen can be used in the composition of the inventions. Optionally, the lectins can be modified with desired haptens such as for example, DNP.

A representative listing of lectins, the abbreviations by which they are referred to, and their sources is given in Table 1.

TABLE 1
Lectins and Abbreviations

| Lectin | Source |
|---|---|
| AAnA | Anguilla anguilla (Eel serum) |
| AAurA | Aleuria aurantia (Orange peel fungus) |
| ABA | Agaricus bisporus (Mushroom) |
| ABrA | Amphicarpanea bracteata (hog-peanut) |
| AL | Hippaestrum hybrid (Amaryllis bulbs) |
| APA | Abrus precatorius (Jequirity bean) |
| BPA | Bauhinia purpurea alba (camel's foot tree) |
| CAA | Caragana arborescens (Siberian pea tree) |
| ConA | Concanavalia ensiformis (Jack bean) |
| CPA | Cicer arietinum (chick pea) |
| CSA | Cytisus scoparius (Scotch broom) |
| DBA | Colichos biflorus (horse gram) |
| DSA | Datura Stramonium (Jimson weed, Thorn apple) |
| ECA | Erythrina crystagalli (Coral tree) |
| ECorA | Erythrina coralldendron (Coral tree) |
| EEA | Euonymus europaeus (spindle tree) |
| DBA | Dolichos biflorus (horse gram) |
| GNA | Galanthus nivalis (Snowdrop bulb) |
| GSA-1/GSA-1I | Griffonia simplicifolia |
| HAA | Helix aspersa (Garden snail) |
| HPA | Helix pomatia (Roman or edible snail) |
| JAC (Jacalin) | Artocarpus integrifolia (jackfruit) |
| LAA | Laburnum alpinum |
| LBA | Phaseolus lunatis (also limensis) (Lima bean) |
| LCA (LcH) | Lens culinaris (lentil) |
| LEA | Lycopersicon esculentum (Tomato) |
| LOA | Lathyrus oderatus (Sweet pea) |
| LTA (LOTUS) | Lotus tetragonolobus (Asparagus pea) |
| MAA | Maackla amurensis (maackla) |
| MPA | Maclura pomifera (Osage orange) |
| NPL (NPA) | Narcissus pseudonarcissus (daffodil) |
| PAA | Phytolacca americana (Pokeweed) |
| PHA (PHA-L) | Phaseolis vulgaris (Red kidney bean) |
| PNA | Arachis hypogaea (Peanut) |
| PSA | Pisum sativum (Pea) |
| PWA | Phytolacca americana (pokeweed) |
| PTAgalactose | Psophocarpus tetagonolobus (winged bean) |
| PTAgalNac | Psophocarpus tetagonolobus (winged bean) |
| RCA-I/RCA-II | Ricinus communis (Castor bean) |
| RPA | Robinia pseudoaccacia (black locust) |
| SBA | Glycine max (Soybean) |
| SJA | Sophora japonica (Japanese pagoda tree) |
| STA | Solanum tuberosum (Potato) |
| TKA | Trichosanthes kinlowii (China gourd) |
| UEA-I/UEA-I1 | Ulex europaeus (Gorse or Furz seeds) |
| VAA | Viscum album (European mistletoe) |
| VFA | Vicia faba (Fava bean) |
| VGA | Vicia graminea |
| VRA | Vigna radiata (mung bean) |
| VSA | Vicia Sativa |
| VVA | Vicia villosa (Hairy vetch) |
| WFA | Wisteria floribunda (Japanese wisteria) |
| WGA | Triticum vulgaris (Wheat germ) |
| suc-WGA | Succinyl WGA |

Thus, in accordance with the invention, the lectin Helix pomatia can be substituted with one or more lectins and/or used in addition with one or more lectins. Therefore, the compositions can be tailored for preventative, therapy and treatment of a wide variety of disorders. For example, N. gonorrheae is agglutinated by lectins that bind to N-acetyl-D-glucosamine residues on their surfaces. Such lectins include WGA and STA, which are known to agglutinate all 193 clinical isolates of N. gonorrheae. WGA is effective for such agglutination at a concentration of 3.1 micrograms per milliliter. Other lectins showing some agglutination activity with respect to N. gonorrheae include RCA-I, RCA-II, GSA-I, and SBA.

Inhibition of Immunoglobulin Isoclass Switching

In a preferred embodiment, the compositions inhibit class switching of IgM to IgG. Macroglobulin, or immunoglobulin-M, is generated by the B1 lymphocyte independent of the T lymphocyte. It is also the first class of globulins made when T and B-lymphocytes interact, but it is quickly replaced by gamma globulin in a process called “isoclass switching.” The most dramatic forms of tissue rejection, such as the blood group incompatibility reaction and hyperacute xenograft rejection, are mediated by macroglobulins. The main advantages of the invention is that by inhibiting isoclass switching from IgM to IgG, the complement cascade is efficiently activated. It takes at least two molecules of IgG to fix the first molecule of complement (C1q). In contrast, a single molecule of IgM can fix C1q and initiate the complement cascade leading to the lysis of the cell.

IgM antibodies are capable of destroying a ‘foreign’ cell. Since there are multiple tumor antigens, all with differing protein structure, an antibody specific for one would necessarily be wrong for the others but what conventional wisdom misses is that tumor antigens are not just proteins; they are glycoproteins.

In another preferred embodiment, the compositions minimize the amount of IgG formed. Unmodified PWM produces IgG and IgM in roughly equivalent amounts. Modification with polyethylene glycol (PEG) or dinitrophenol decreases IgG production. We were able to substantially decrease IgG production by coating 50% of the molecule with PEG.

Cancer Therapy

In accordance with the invention tumor target cells are selectively targeted by the compositions by, for example, inclusion of antibodies specific for an antigen. Tumor antigens can be the result of infection by a tumor causing virus and the viral antigens expressed on the surface of an infected cell could be targeted using this technology. Non-limiting examples of tumor antigens, include, tumor antigens resulting from mutations, such as: alpha-actinin-4 (lung carcinoma); BCR-ABL fusion protein (b3a2) (chronic myeloid leukemia); CASP-8 (head and neck squamous cell carcinoma); beta-catenin (melanoma); Cdc27 (melanoma); CDK4 (melanoma); dek-can fusion protein (myeloid leukemia); Elongation factor 2 (lung squamous carcinoa); ETV6-AML1 fusion protein (acute lymphoblastic leukemia); LDLR-fucosyltransferaseAS fusion protein (melanoma); overexpression of HLA-A2^{d} (renal cell carcinoma); hsp70-2 (renal cell carcinoma); KIAA0205 (bladder tumor); MART2 (melanoma); MUM-lf(melanoma); MUM-2 (melanoma); MUM-3 (melanoma); neo-PAP (melanoma); Myosin class I (melanoma); OS-9g (melanoma); pml-RARalpha fusion protein (promyelocytic leukemia); PTPRK (melanoma); K-ras (pancreatic adenocarcinoma); N-ras (melanoma). Examples of differentiation tumor antigens include, but not limited to: CEA (gut carcinoma); gp110/Pme117 (melanoma); Kallikrein 4 (prostate); mammaglobin-A (breast cancer); Melan-A /MART-1 (melanoma); PSA (prostate carcinoma); TRP-1/gp75 (melanoma); TRP-2 (melanoma); tyrosinase (melanoma). Over or under-expressed tumor antigens include but are not limited to: CPSF (ubiquitous); EphA3; G250/MN/CAIX (stomach, liver, pancreas); HER-2/neu; Intestinal carboxyl esterase (liver, intestine, kidney); alpha-foetoprotein (liver); M-CSF (liver, kidney); MUCI (glandular epithelia); p53 (ubiquitous); PRAME (testis, ovary, endometrium, adrenals); PSMA (prostate, CNS, liver); RAGE-1 (retina); RU2AS (testis, kidney, bladder); survivin (ubiquitous); Telomerase (testis, thymus, bone marrow, lymph nodes); WT1 (testis, ovary, bone marrow, spleen); CA125 (ovarian).

The invention may be used against protein coding gene products as well as non-protein coding gene products. Examples of non-protein coding gene products include gene products that encode ribosomal RNAs, transfer RNAs, small nuclear RNAs, small cytoplasmic RNAs, telomerase RNA, RNA molecules involved in DNA replication, chromosomal rearrangement and the like.

In another preferred embodiment, abnormal or cancer cells are targeted by the compositions. For example, many malignancies are associated with the presence of foreign DNA, e.g. Bcr-Ab1, Bcl-2, HPV, and these provide unique molecular targets e.g. antigens, to permit selective malignant cell targeting. The approach can be used to target expression products as a result of single base substitutions (e.g. K-ras, p53) or methylation changes. However, proliferation of cancer cells may also be caused by previously unexpressed gene products. These gene sequences can be targeted, thereby, inhibiting further expression and ultimate death of the cancer cell. In other instances, transposons can be the cause of such deregulation and transposon sequences can be targeted, e.g. Tn5.

The invention in general provides a method for treating diseases, such as cancer and diseases which are caused by infectious agents such as viruses, bacteria, intra- and extra-cellular parasites, insertion elements, fungal infections, etc., which may also cause expression of gene products by a normally unexpressed gene, abnormal expression of a normally expressed gene or expression of an abnormal gene.

The methods of the invention are preferably employed for treatment or prophylaxis against diseases caused abnormal cell growth and by infectious agents, particularly for treatment of infections as may occur in tissue such as lung, heart, liver, prostate, brain, testes, stomach, intestine, bowel, spinal cord, sinuses, urinary tract or ovaries of a subject. The methods of the invention also may be employed to treat systemic conditions such as viremia or septicemia. The methods of the invention are also preferably employed for treatment of diseases and disorders associated with viral infections or bacterial infections, as well as any other disorder caused by an infectious agent.

In another preferred embodiment, the compositions of the invention can be administered in conjunction with chemotherapy. These chemotherapeutic agents can be co-administered, precede, or administered after the compositions. Non-limiting examples of chemotherapeutic agents include, but not limited to: cyclophosphamide (CTX 25 mg/kg/day,p.o.), taxanes (paclitaxel or docetaxel), busulfan, cisplatin, cyclophosphamide, methotrexate, daunorubicin, doxorubicin, melphalan, cladribine, vincristine, vinblastine, and chlorambucil.

In another preferred embodiment, the pharmaceutical composition, inhibits the tumor cell growth in a subject, and the method comprises administering to the subject a pharmaceutical composition comprising a therapeutically effective amount of the composition. Inhibition of tumor cell growth refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

Combination Therapies

The PWM-poly IC or PWM -PHA-poly IC therapeutic compositions of the present invention may be combined with any other methods generally employed in the treatment of the particular tumor, disease or disorder that the patient exhibits. So long as a particular therapeutic approach is not known to be detrimental to the patient's condition in itself, and does not significantly counteract the PWM-poly IC composition treatment, its combination with the present invention is contemplated.

In connection solid tumor treatment, the present invention may be used in combination with classical approaches, such as surgery, radiotherapy, chemotherapy, and the like. The invention therefore provides combined therapies in which the PWM-poly IC or PWM-PHA-poly IC therapeutic compositions are used simultaneously with, before, or after surgery or radiation treatment; or are administered to patients with, before, or after conventional chemotherapeutic, radiotherapeutic or other anti-angiogenic agents, or targeted immunotoxins or coaguligands.

Combination therapy for other vascular diseases is also contemplated. A particular example of such is benign prostatic hyperplasia (BPH), which may be treated with PWM-poly IC or PWM-PHA-poly IC compositions in combination other treatments currently practiced in the art. For example, targeting of immunotoxins to markers localized within BPH, such as PSA.

When one or more agents are used in combination with the PWM-poly IC or PWM-PHA-poly IC compositions, there is no requirement for the combined results to be additive of the effects observed when each treatment is conducted separately. Although at least additive effects are generally desirable, any increased anti-tumor effect above one of the single therapies would be of benefit. Also, there is no particular requirement for the combined treatment to exhibit synergistic effects, although this is certainly possible and advantageous.

To practice combined anti-tumor therapy, one would simply administer to an animal the PWM-poly IC or PWM-PHA-poly IC composition construct in combination with another anti-cancer agent in a manner effective to result in their combined anti-tumor actions within the animal. The agents would therefore be provided in amounts effective and for periods of time effective to result in their combined presence within the tumor vasculature and their combined actions in the tumor environment. To achieve this goal, the PWM-poly IC or PWM-PHA-poly IC compositions and other anti-cancer agents may be administered to the animal simultaneously, either in a single composition, or as two distinct compositions using different administration routes.

Alternatively, the PWM-poly IC or PWM-PHA-poly IC composition mediated treatment may precede, or follow, the a second anti-cancer agent treatment by, e.g., intervals ranging from minutes to weeks. In certain embodiments where the anti-cancer agent and the PWM-poly IC or PWM-PHA-poly IC composition are applied separately to the animal, one would ensure that a significant period of time did not expire between the time of each delivery, such that the anti-cancer agent and the PWM-poly IC or PWM-PHA-poly IC composition would still be able to exert an advantageously combined effect on the tumor. In such instances, it is contemplated that one would contact the tumor with both agents within about 5 minutes to about one week of each other and, more preferably, within about 12-72 hours of each other, with a delay time of only about 12-48 hours being most preferred.

The general use of combinations of substances in cancer treatment is well known. For example, U.S. Pat. No. 5,710,134 (incorporated herein by reference) discloses components that induce necrosis in tumors in combination with non-toxic substances or “prodrugs”. The enzymes set free by necrotic processes cleave the non-toxic “prodrug” into the toxic “drug”, which leads to tumor cell death. Also, U.S. Pat. No. 5,747,469 (incorporated herein by reference) discloses the combined use of viral vectors encoding p53 and DNA damaging agents. Any such similar approaches can be used with the present invention.

In some situations, it may even be desirable to extend the time period for treatment significantly, where several days (2, 3, 4, 5, 6 or 7), several weeks (1, 2, 3, 4, 5, 6, 7 or 8) or even several months (1, 2, 3, 4, 5, 6, 7 or 8) lapse between the respective administrations. This would be advantageous in circumstances where one treatment was intended to substantially destroy the tumor, such as the PWM-poly IC or PWM-PHA-poly IC composition treatment, and another treatment was intended to prevent micrometastasis or tumor re-growth, such as the administration of an anti-angiogenic agent.

It also is envisioned that more than one administration of either the PWM-poly IC or PWM-PHA-poly IC composition or another anti-cancer agent will be utilized. The PWM-poly IC or PWM-PHA-poly IC composition and anti-cancer agents may be administered interchangeably, on alternate days or weeks; or a sequence of the PWM-poly IC or PWM-PHA-poly IC composition treatment may be given, followed by a sequence of anti-cancer agent therapy. In any event, to achieve tumor regression using a combined therapy, all that is required is to deliver both agents in a combined amount effective to exert an anti-tumor effect, irrespective of the times for administration.

In terms of surgery, any surgical intervention may be practiced in combination with the present invention. In connection with radiotherapy, any mechanism for inducing DNA damage locally within tumor cells is contemplated, such as γ-irradiation, X-rays, UV-irradiation, microwaves and even electronic emissions and the like. The directed delivery of radioisotopes to tumor cells is also contemplated, and this may be used in connection with a targeting antibody or other targeting means.

Cytokine therapy also has proven to be an effective partner for combined therapeutic regimens. Various cytokines may be employed in such combined approaches. Examples of cytokines include IL-1α, IL-1β, IL-2, IL-3, IL-4, IL-5, IL-6, IL-7, IL-8, IL-9, IL-10, IL-11, IL-12, IL-13, TGF-β, GM-CSF, M-CSF, G-CSF, TNFα, TNFβ, LAF, TCGF, BCGF, TRF, BAF, BDG, MP, LIEF, OSM, TMF, PDGF, IFN-α, IFN-β, IFN-γ. Cytokines are administered according to standard regimens, consistent with clinical indications such as the condition of the patient and relative toxicity of the cytokine. Uteroglobins may also be used to prevent or inhibit metastases (U.S. Pat. No. 5,696,092; incorporated herein by reference).

PWM-Poly IC-PHA Compositions and Combination Chemotherapeutics

In certain embodiments, the PWM-poly IC or PWM-PHA-poly IC composition of the present invention may be administered in combination with another chemotherapeutic agent. Irrespective of the underlying mechanism(s), a variety of chemotherapeutic agents may be used in the combined treatment methods disclosed herein. Therapeutic agents can include, for example, chemotherapeutic agents such as, cyclophosphamide (CTX, 25 mg/kg/day, p.o.), taxanes (paclitaxel or docetaxel), busulfan, cisplatin, methotrexate, daunorubicin, doxorubicin, melphalan, cladribine, vincristine, vinblastine, chlorambucil, tamoxifen, taxol, etoposide (VP-16), adriamycin, 5-fluorouracil (5FU), camptothecin, actinomycin-D, mitomycin C, cisplatin (CDDP), combretastatin(s) and derivatives and prodrugs thereof.

As will be understood by those of ordinary skill in the art, the appropriate doses of chemotherapeutic agents will be generally around those already employed in clinical therapies wherein the chemotherapeutics are administered alone or in combination with other chemotherapeutics. By way of example only, agents such as cisplatin, and other DNA alkylating may be used. Cisplatin has been widely used to treat cancer, with efficacious doses used in clinical applications of 20 mg/m² for 5 days every three weeks for a total of three courses. Cisplatin is not absorbed orally and must therefore be delivered via injection intravenously, subcutaneously, intratumorally or intraperitoneally.

Further useful agents include compounds that interfere with DNA replication, mitosis and chromosomal segregation. Such chemotherapeutic compounds include adriamycin, also known as doxorubicin, etoposide, verapamil, podophyllotoxin, and the like. Widely used in a clinical setting for the treatment of neoplasms, these compounds are administered through bolus injections intravenously at doses ranging from 25-75 mg/m² at 21 day intervals for adriamycin, to 35-50 mg/m² for etoposide intravenously or double the intravenous dose orally.

Agents that disrupt the synthesis and fidelity of polynucleotide precursors may also be used. Particularly useful are agents that have undergone extensive testing and are readily available. As such, agents such as 5-fluorouracil (5-FU) are preferentially used by neoplastic tissue, making this agent particularly useful for targeting to neoplastic cells. Although quite toxic, 5-FU, is applicable in a wide range of carriers, including topical, however intravenous administration with doses ranging from 3 to 15 mg/kg/day being comrnonly used.

The skilled artisan is directed to “Remington's Pharmaceutical Sciences” 15th Edition, chapter 33, in particular pages 624-652, for non-limiting examples of other chemotherapeutic agents that can be used in combination therapies with the PWM-poly IC-PHA compositions. Some variation in dosage will necessarily occur depending on the condition of the subject being treated. The physician responsible for administration will be able to determine the appropriate dose for the individual subject.

PWM-Poly IC-PHA Compositions Combination Therapy with Anti-Angiogenics

In a preferred embodiment, the PWM-poly IC or PWM-PHA-poly IC compositions are administered in combination with anti-angiogenics. The term “angiogenesis” refers to the generation of new blood vessels, generally into a tissue or organ. Under normal physiological conditions, humans or animals undergo angiogenesis only in very specific restricted situations. For example, angiogenesis is normally observed in wound healing, fetal and embryonic development and formation of the corpus luteum, endometrium and placenta. Uncontrolled (persistent and/or unregulated) angiogenesis is related to various disease states, and occurs during tumor growth and metastasis.

Both controlled and uncontrolled angiogenesis are thought to proceed in a similar manner. Endothelial cells and pericytes, surrounded by a basement membrane, form capillary blood vessels. Angiogenesis begins with the erosion of the basement membrane by enzymes released by endothelial cells and leukocytes. The endothelial cells, which line the lumen of blood vessels, then protrude through the basement membrane. Angiogenic stimulants induce the endothelial cells to migrate through the eroded basement membrane. The migrating cells form a “sprout” off the parent blood vessel, where the endothelial cells undergo mitosis and proliferate. The endothelial sprouts merge with each other to form capillary loops, creating the new blood vessel.

As persistent, unregulated angiogenesis occurs during tumor development and metastasis, the treatment methods of this invention may be used in combination with any one or more “anti-angiogenic” therapies. Exemplary anti-angiogenic agents that are useful in connection with combined therapy are listed in Table 2. Each of the agents listed therein is exemplary and by no means limiting.

TABLE 2
Inhibitors and Negative Regulators of Angiogenesis

Substances
Angiostatin
Endostatin
16 kDa prolactin fragment
Laminin peptides
Fibronectin peptides
Tissue metalloproteinaseinhibitors (TIMP 1, 2, 3, 4)
Plasminogen activator inhibitors (PAI-1, -2)
Tumor necrosis factor alpha (high dose, in vitro)
TGF-β1
Interferons (IFN-α, -β, -γ)
ELR- CXC Chemokines:IL-12; SDF-1; MIG; Platelet factor
4 (PF-4);
IP-10
Thrombospondin (TSP)
SPARC
2-Methoxyoestradiol
Proliferin-related protein
Suramin
Thalidomide
Cortisone
Fumagillin (AGM-1470; TNP-470)
Tamoxifen
Korean mistletoe extract (Viscum album coloratum)
Retinoids
CM101
Dexamethasone
Leukemia inhibitory factor (LIF)

A certain preferred component for use in inhibiting angiogenesis is a protein named “angiostatin”. This component is disclosed in U.S. Pat. Nos. 5,776,704; 5,639,725 and 5,733,876, each incorporated herein by reference. Angiostatin is a protein having a molecular weight of between about 38 kD and about 45 kD, as determined by reducing polyacrylamide gel electrophoresis, which contains approximately Kringle regions 1 through 4 of a plasminogen molecule. Angiostatin generally has an amino acid sequence substantially similar to that of a fragment of murine plasminogen beginning at amino acid number 98 of an intact murine plasminogen molecule.

The amino acid sequence of angiostatin varies slightly between species. For example, in human angiostatin, the amino acid sequence is substantially similar to the sequence of the above described murine plasminogen fragment, although an active human angiostatin sequence may start at either amino acid number 97 or 99 of an intact human plasminogen amino acid sequence. Further, human plasminogen may be used, as it has similar anti-angiogenic activity, as shown in a mouse tumor model.

Certain anti-angiogenic therapies have already been shown to cause tumor regressions, and angiostatin is one such agent. Endostatin, a 20 kDa COOH-terminal fragment of collagen XVIII, the bacterial polysaccharide CM101, and the antibody LM609 also have angiostatic activity. However, in light of their other properties, they are referred to as anti-vascular therapies or tumor vessel toxins, as they not only inhibit angiogenesis but also initiate the destruction of tumor vessels through mostly undefined mechanisms. Their combination with the present invention is clearly envisioned.

Angiostatin and endostatin have become the focus of intense study, as they are the first angiogenesis inhibitors that have demonstrated the ability to not only inhibit tumor growth but also cause tumor regressions in mice. There are multiple proteases that have been shown to produce angiostatin from plasminogen including elastase, macrophage metalloelastase (MME), matrilysin (MMP-7), and 92 kDa gelatinase B/type IV collagenase (MMP-9).

MME can produce angiostatin from plasminogen in tumors and granulocyte-macrophage colony-stimulating factor (GMCSF) upregulates the expression of MME by macrophages inducing the production of angiostatin. The role of MME in angiostatin generation is supported by the finding that MME is in fact expressed in clinical samples of hepatocellular carcinomas from patients. Another protease thought to be capable of producing angiostatin is stromelysin-1 (MMP-3). MMP-3 has been shown to produce angiostatin-like fragments from plasminogen in vitro.

CM101 is a bacterial polysaccharide that has been well characterized in its ability to induce neovascular inflammation in tumors. CM101 binds to and cross-links receptors expressed on dedifferentiated endothelium that stimulates the activation of the complement system. It also initiates a cytokine-driven inflammatory response that selectively targets the tumor. It is an anti-pathoangiogenic agent that downregulates the expression VEGF and its receptors.

Thrombospondin (TSP-1) and platelet factor 4 (PF4) may also be used in combination with the present invention. These are both angiogenesis inhibitors that associate with heparin and are found in platelet α-granules. TSP-1 is a large 450 kDa multi-domain glycoprotein that is constituent of the extracellular matrix. TSP-1 binds to many of the proteoglycan molecules found in the extracellular matrix including, HSPGs, fibronectin, laminin, and different types of collagen. TSP-1 inhibits endothelial cell migration and proliferation in vitro and angiogenesis in vivo. TSP-1 can also suppress the malignant phenotype and tumorigenesis of transformed endothelial cells. The tumor suppressor gene p53 has been shown to directly regulate the expression of TSP-1 such that, loss of p53 activity causes a dramatic reduction in TSP-1 production and a concomitant increase in tumor initiated angiogenesis.

PF4 is a 70 amino acid protein that is member of the CXC ELR-family of chemokines that is able to potently inhibit endothelial cell proliferation in vitro and angiogenesis in vivo. PF4 administered intratumorally or delivered by an adenoviral vector is able to cause an inhibition of tumor growth.

Interferons and metalloproteinase inhibitors are two other classes of naturally occurring angiogenic inhibitors that can be combined with the present invention. The anti-endothelial activity of the interferons has been known since the early 1980s, however, the mechanism of inhibition is still unclear. It is known that they can inhibit endothelial cell migration and that they do have some anti-angiogenic activity in vivo that is possibly mediated by an ability to inhibit the production of angiogenic promoters by tumor cells. Vascular tumors in particular are sensitive to interferon, for example, proliferating hemangiomas can be treated with IFNα.

Tissue inhibitors of metalloproteinases (TIMPs) are a family of naturally occurring inhibitors of matrix metalloproteases (MMPs) that can also inhibit angiogenesis and can be used in combined treatment protocols with the present invention. MMPs play a key role in the angiogenic process as they degrade the matrix through which endothelial cells and fibroblasts migrate when extending or remodeling the vascular network. In fact, one member of the MMPs, MMP-2, has been shown to associate with activated endothelium through the integrin αvβ3 presumably for this purpose. If this interaction is disrupted by a fragment of MMP-2, then angiogenesis is downregulated and in tumors growth is inhibited.

There are a number of pharmacological agents (anti-angiogenics) that inhibit angiogenesis, any one or more of which may be used in combination with the present invention. These include AGM-1470/TNP-470, thalidomide, and carboxyamidotriazole (CAI). Funagillin was found to be a potent inhibitor of angiogenesis in 1990, and since then the synthetic analogues of funagillin, AGM-1470 and TNP-470 have been developed. Both of these drugs inhibit endothelial cell proliferation in vitro and angiogenesis in vivo. TNP-470 has been studied extensively in human clinical trials with data suggesting that long-term administration is optimal.

Thalidomide was originally used as a sedative but was found to be a potent teratogen and was discontinued. In 1994 it was found that thalidomide is an angiogenesis inhibitor. Thalidomide is currently in clinical trials as an anti-cancer agent as well as a treatment of vascular eye diseases.

CAI is a small molecular weight synthetic inhibitor of angiogenesis that acts as a calcium channel blocker that prevents actin reorganization, endothelial cell migration and spreading on collagen IV. CAI inhibits neovascularization at physiological attainable concentrations and is well tolerated orally by cancer patients. Clinical trials with CAI have yielded disease stabilization in 49% of cancer patients having progressive disease before treatment.

Cortisone in the presence of heparin or heparin fragments was shown to inhibit tumor growth in mice by blocking endothelial cell proliferation. The mechanism involved in the additive inhibitory effect of the steroid and heparin is unclear although it is thought that the heparin may increase the uptake of the steroid by endothelial cells. The mixture has been shown to increase the dissolution of the basement membrane underneath newly formed capillaries and this is also a possible explanation for the additive angiostatic effect. Heparin-cortisol conjugates also have potent angiostatic and anti-tumor effects activity in vivo.

Further specific angiogenesis inhibitors, including, but not limited to, Anti-Invasive Factor, retinoic acids and paclitaxel (U.S. Pat. No. 5,716,981; incorporated herein by reference); AGM-1470 (Ingber et al., Nature, 48:555-557 1990; incorporated herein by reference); shark cartilage extract (U.S. Pat. No. 5,618,925; incorporated herein by reference); anionic polyamide or polyurea oligomers (U.S. Pat. No. 5,593,664; incorporated herein by reference); oxindole derivatives (U.S. Pat. No. 5,576,330; incorporated herein by reference); estradiol derivatives (U.S. Pat. No. 5,504,074; incorporated herein by reference); and thiazolopyrimidine derivatives (U.S. Pat. No. 5,599,813; incorporated herein by reference) are also contemplated for use as anti-angiogenic compositions for the combined uses of the present invention.

Compositions comprising an antagonist of an αᵥβ₃ integrin may also be used to inhibit angiogenesis in combination with the present invention. As disclosed in U.S. Pat. No. 5,766,591 (incorporated herein by reference), RGD-containing polypeptides and salts thereof, including cyclic polypeptides, are suitable examples of αᵥβ₃ integrin antagonists.

The antibody LM609 against the αᵥβ₃ integrin also induces tumor regressions. Integrin αᵥβ₃ antagonists, such as LM609, induce apoptosis of angiogenic endothelial cells leaving the quiescent blood vessels unaffected. LM609 or other αᵥβ₃ antagonists may also work by inhibiting the interaction of αᵥβ₃ and MMP-2, a proteolytic enzyme thought to play an important role in migration of endothelial cells and fibroblasts.

Apoptosis of the angiogenic endothelium in this case may have a cascade effect on the rest of the vascular network. Inhibiting the tumor vascular network from completely responding to the tumor's signal to expand may, in fact, initiate the partial or full collapse of the network resulting in tumor cell death and loss of tumor volume. It is possible that endostatin and angiostatin function in a similar fashion. The fact that LM609 does not affect quiescent vessels but is able to cause tumor regressions suggests strongly that not all blood vessels in a tumor need to be targeted for treatment in order to obtain an anti-tumor effect.

Non-targeted angiopoietins, such as angiopoietin-2, may also be used in combination with the present invention. The angiogenic effects of various regulators involve an autocrine loop connected with angiopoietin-2. The use of angiopoietin-2, angiopoietin-1, angiopoietin-3 and angiopoietin-4, is thus contemplated in conjunction with the present invention. Other methods of therapeutic intervention based upon altering signaling through the Tie2 receptor can also be used in combination herewith, such as using a soluble Tie2 receptor capable of blocking Tie2 activation (Lin et al., Proc. Natl. Acad. Sci., USA, 95(15):8829-34, 1998). Delivery of such a construct using recombinant adenoviral gene therapy has been shown to be effective in treating cancer and reducing metastases (Lin et al., 1998).

PWM-Poly IC-PHA Compositions and Combination Therapy with Apoptosis-Inducing Agents

The PWM-poly IC or PWM-PHA-poly IC composition treatment may also be combined with treatment methods that induce apoptosis in any cells within the tumor, including tumor cells and tumor vascular endothelial cells. Although many anti-cancer agents may have, as part of their mechanism of action, an apoptosis-inducing effect, certain agents have been discovered, designed or selected with this as a primary mechanism, as described below.

A number of oncogenes have been described that inhibit apoptosis, or programmed cell death. Exemplary oncogenes in this category include, but are not limited to, bcr-abl, bcl-2 (distinct from bcl-1, cyclin D1; GenBank accession numbers M14745, X06487; U.S. Pat. Nos. 5,650,491; and 5,539,094; each incorporated herein by reference) and family members including Bcl-xl, Mcl-1, Bak, A1, A20. Overexpression of bcl-2 was first discovered in T cell lymphomas. bcl-2 functions as an oncogene by binding and inactivating Bax, a protein in the apoptotic pathway. Inhibition of bcl-2 function prevents inactivation of Bax, and allows the apoptotic pathway to proceed. Thus, inhibition of this class of oncogenes, e.g., using antisense nucleotide sequences, is contemplated for use in the present invention in aspects wherein enhancement of apoptosis is desired (U.S. Pat. Nos. 5,650,491; 5,539,094; and 5,583,034; each incorporated herein by reference).

Many forms of cancer have reports of mutations in tumor suppressor genes, such as p53. Inactivation of p53 results in a failure to promote apoptosis. With this failure, cancer cells progress in tumorigenesis, rather than become destined for cell death. Thus, provision of tumor suppressors is also contemplated for use in the present invention to stimulate cell death. Exemplary tumor suppressors include, but are not limited to, p53, Retinoblastoma gene (Rb), Wilm's tumor (WT 1), bax alpha, interleukin-1β-converting enzyme and family, MEN-1 gene, neurofibromatosis, type 1 (NF1), cdk inhibitor p16, colorectal cancer gene (DCC), familial adenomatosis polyposis gene (FAP), multiple tumor suppressor gene (MTS-1), BRCA1 and BRCA2.

Preferred for use are the p53 (U.S. Pat. Nos. 5,747,469; 5,677,178; and 5,756,455; each incorporated herein by reference), Retinoblastoma, BRCA1 (U.S. Pat. Nos. 5,750,400; 5,654,155; 5,710,001; 5,756,294; 5,709,999; 5,693,473; 5,753,441; 5,622,829; and 5,747,282; each incorporated herein by reference), MEN-1 (GenBank accession number U93236) and adenovirus EIA (U.S. Pat. No. 5,776,743; incorporated herein by reference) genes.

Other compositions that may be used include genes encoding the tumor necrosis factor related apoptosis inducing ligand termed TRAIL, and the TRAIL polypeptide (U.S. Pat. No. 5,763,223; incorporated herein by reference); the 24 kD apoptosis-associated protease of U.S. Pat. No. 5,605,826 (incorporated herein by reference); Fas-associated factor 1, FAF1 (U.S. Pat. No. 5,750,653; incorporated herein by reference). Also contemplated for use in these aspects of the present invention is the provision of interleukin-1β-converting enzyme and family members, which are also reported to stimulate apoptosis.

Compounds such as carbostyril derivatives (U.S. Pat. Nos. 5,672,603; and 5,464,833; each incorporated herein by reference); branched apogenic peptides (U.S. Pat. No. 5,591,717; incorporated herein by reference); phosphotyrosine inhibitors and non-hydrolyzable phosphotyrosine analogs (U.S. Pat. Nos. 5,565,491; and 5,693,627; each incorporated herein by reference); agonists of RXR retinoid receptors (U.S. Pat. No. 5,399,586; incorporated herein by reference); and even antioxidants (U.S. Pat. No. 5,571,523; incorporated herein by reference) may also be used. Tyrosine kinase inhibitors, such as genistein, may also be linked to ligands that target a cell surface receptor (U.S. Pat. No. 5,587,459; incorporated herein by reference).

Effective Amounts

The compositions described above are preferably administered to a subject in an effective amount. An effective amount is an amount which is capable of producing a desirable: result in a treated animal or cell (for example, to induce apoptosis or impair mitosis in a cell in the animal or a culture). As is well known in the medical and veterinary arts, dosage for any one animal depends on many factors, including the particular animal's size, body surface area, age, the particular composition to be administered, time and route of administration, general health, and other drugs being administered concurrently. It is expected that an appropriate dosage for topical administration of the compositions of the invention would be in the range of about 1.5-4.0 mg PWM-poly IC-PHA/kg of body weight (e.g., 200 mg for subjects ranging from 110 to 300 lbs). An effective amount for use with a cell in culture will also vary, but can be readily determined empirically (for example, by adding varying concentrations to the cell and selecting the concentration that best produces the desired result). It is expected that an appropriate concentration would be in the range of about 5-200 μM.

Method for Inhibiting Cancer Cell Growth

The invention provides a method for inhibiting tumor cell growth or increasing the rate of tumor cell apoptosis. The method includes the steps of contacting a tumor cell with a composition including a sufficient amount of PWM-poly IC or PWM-PHA-poly IC compositions to kill or at least retard mitosis in the tumor cell. The method may be used to inhibit the growth of numerous types of cancerous tumor cells. PWM-poly IC or PWM-PHA-poly IC has been tested and shown to be effective against different types of tumors such as melanoma, squamous, and breast cancer cells. PWM-poly IC or PWM-PHA-poly IC compositions are expected to be effective against other cancers as well, particularly those derived from epithelial, mesenchymal, and hemopoietic origins.

Any suitable formulation of PWM-poly IC or PWM-PHA-poly IC can be used in methods of the invention. Typical formulations are topical liposomal formulations of PWM-poly IC or PWM-PHA-poly IC of varying concentrations. In addition to topical administration, PWM-poly IC or PWM-PHA-poly IC -containing formulations can be administered to a subject via injection (e.g., IP, IV, IM, SQ).

In a method of reducing the rate of tumor cell growth or increasing the rate of tumor cell apoptosis in vitro, PWM-poly IC or PWM-PHA-poly IC is dissolved in a suitable medium such as for example, 2-propanol followed by dilution in a desired medium. In an in vivo method of reducing the rate of tumor cell growth or increasing the rate of tumor cell apoptosis, a PWM-poly IC or PWM-PHA-poly IC—containing cream is applied topically daily to the target site until tumor regression occurs. In another in vivo method, a PWM-poly IC or PWM-PHA-poly IC—containing formulation is administered to a subject via injection (e.g., IP, IV, IM, SQ).

Inhibition of tumor cell growth manifested by administration of the PWM-poly IC or PWM-PHA-poly IC compositions described herein, that is compositions comprising about 1% to about 25% coenzyme Q10, refers to one or more of the following effects: (1) inhibition, to some extent, of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

In preferred embodiments, administration of PWM-poly IC or PWM-PHA-poly IC compositions results in one or more phenotypes of a tumor cell being inhibited. For example, inhibition of tumor growth, reduction of tumor size, inhibition of metastasis, reduction in the number of tumor cells and the like. Each of these phenotypes of a tumor cell can be measured using standard assays, such as for example, imaging, mechanical measurements, in vitro assays and the like.

Imaging Techniques

In a preferred embodiment, the imaging technique is magnetic resonance imaging (MRI). Magnetic resonance imaging (MRI) is used for producing images of the body in a variety of scanning planes such as, for example, axial, coronal, sagittal or orthogonal. MRI employs a magnetic field, radio frequency energy and magnetic field gradients to make images of the body. Similar to CTs, the magnetic resonance imaging techniques which are employed are conventional and are described, for example, in D. M. Kean and M. A. Smith, Magnetic Resonance Imaging: Principles and Applications, (William and Wilkins, Baltimore 1986), and in Rajan, S. S., MRI: A Conceptual Overview (Springer Verlag 1997), the disclosures of which are incorporated by reference herein in their entirety. Contemplated MRI techniques include, but are not limited to, nuclear magnetic resonance (NMR), electronic spin resonance (ESR), and the like.

In accordance with the present invention, any imaging techniques which allow for the monitoring of the labeled PWM-poly IC or PWM-PHA-poly IC intb the target region are compatible with the teachings herein. In this regard, all forms of imaging techniques are contemplated in the present invention, including, for example, imaging by X-ray, computed tomography (CT) imaging, including CT angiography (CTA) imaging, magnetic resonance (MR) imaging, magnetic resonance angiography (MRA), nuclear medicine, ultrasound (US) imaging, optical imaging or spectroscopy, elastography, infrared imaging, microwave imaging, and the like. Moreover, the imaging may be combined to provide multiple exposures of the contrast agent following administration. The imaging techniques that are employed are known in the art, and these techniques are described generally in Kopans, D. B. Md., Breast Imaging (Lippincott-Raven Publishers 1998), the disclosure of which is incorporated by reference herein in its entirety.

Computed tomography (CT) is a valuable diagnostic imaging technique for studying various areas of the body. This technique measures the radiodensity (electron density) of matter. CT imaging techniques which are employed are conventional and are described, for example, in Computed Body Tomography, Lee, J. K. T., Sagel, S. S., and Stanley, R. J., eds., 1983, Ravens Press, New York, N.Y., especially the first two chapters thereof entitled “Physical Principles and Instrumentation”, and Scroggin, Lippincotts Computer Tomography Review (Lippincott-Raven Publishers 1995), the disclosures of which are incorporated by reference herein in their entirety.

With respect to ultrasound, ultrasonic imaging techniques contemplated for use in the present invention are well known in the art, and are described, for example, in McGahan and Goldberg, Diagnostic Ultrasound: A Logical Approach (Lippincott-Raven Publishers 1998), and in Frederick and Kremkau, Diagnostic Ultrasound: Principles and Instruments, (W B Saunders Co. 1998), the disclosures of which are hereby incorporated herein by reference in their entirety.

Specific ultrasound imaging modes useful with the disclosed invention include harmonic or non-linear imaging, grey scale (B-mode), Doppler (including pulsed wave, Power, flow, color, amplitude, spectral and harmonic), 3-D imaging, gated imaging, and the like. With respect to harmonic imaging, it will be appreciated that the present invention is compatible with wideband harmonic imaging and pulse inversion harmonic imaging. Those skilled in the art will further appreciate that any of these imaging modes may be used to provide the signal levels which, upon processing, can afford the desired values for fluid flow rates and fluid content.

If one desires to use harmonic imaging (an optional embodiment of the present invention), and the ultrasound imaging machine is set to image at a particular frequency, the outgoing waveform supplied to the sonic transducer can be a numerical fraction of the imaging frequency (e.g., 1/2, 2/3, 1/3, and the like) or a whole number or fractional multiple of the imaging frequency (e.g., 2, 3/2, 3, 4, and the like). With any particular combination of labeled PWM-poly IC or PWM-PHA-poly IC and contrast agents and excitation frequency, certain harmonics will be dominant. The second harmonic is a common example. Those strongest harmonics are preferred for obvious reasons, although other harmonics or frequencies may be selected for reasons such as preparation of multiple images or elimination of background. Moreover several frequencies, including harmonic and non-harmonic frequencies or some combination thereof, may be simultaneously detected to provide the desired image. That is, in preferred embodiments any frequency other than the interrogation frequency may be used to provide the desired data. Of course, those skilled in the art will appreciate that dominant harmonics can be determined by simple empirical testing of the contrast agent preparation.

To detect the reradiated ultrasound energy generated by the contrast agents and PWM-poly IC or PWM-PHA-poly IC, a modified conventional ultrasound scanner system or commercially available harmonic imaging systems can be used. These systems are able to detect or select one or more or all of the new frequencies, or harmonics, radiated by the contrast agents for production of the ultrasound image. In other words, it detects a frequency different from the emitted frequency. Equipment suitable for harmonic ultrasound imaging is disclosed in Williams et al., WO 91/15999. Many conventional ultrasound imaging devices, however, utilize transducers capable of broad bandwidth operation, and the outgoing waveform sent to the transducer is software controlled. For this reason, reprogramming to emit a waveform different from the one detected is well within the level of skill in the art.

Although harmonic ultrasound imaging is preferred for use in the disclosed methods and systems, other types of ultrasound imaging such as B-mode (gray scale imaging), F-mode (color flow or Doppler imaging) and D-mode (spectral Doppler) are also compatible and within the purview of the instant invention.

In B-mode imaging, the ultrasound system typically transmits a series of beams, along scan lines, steered to scan a desired field of view. The ultrasound system typically steers “receive beams” in a manner that corresponds to the transmit beams. Data returned from each receive beam is communicated to an image display subsystem which reconstructs a two-dimensional gray scale image from the B-mode data and displays it on a console. Such series of pulses down a single line may be identical or may be of equal or unequal frequency or have a near 180 degree phase shift (inverted pulse) to promote the distinction of the contrast agent from the surrounding tissues.

F-mode imaging is accomplished in a manner similar to B-mode imaging, in that the ultrasound system fires and receives a series of beams to scan a field of view. However, since F-mode imaging requires calculation of the velocity of targets, each line is fired and received several times. As with B-mode imaging, the data returned from each firing of each line is used to reconstruct an image on a console. F-mode imaging is often used concurrently with B-mode imaging. For example, the gray scale image reconstructed from a B-mode scan can be superimposed with an F-mode image reconstructed from an F-mode scan of the same field of view or of a lesser included field of view. The F-mode information can be displayed using colors, with different colors indicating different positive or negative flow velocities or turbulence at the part of the B-mode image on which the pixel is superimposed. Because F-mode imaging is intended to provide only qualitative insight into target motion in the patient's body, the ultrasound system's processing of F-mode signals need not have high spatial or velocity resolution either in amplitude or in pixel resolution. However, since an important value of F-mode imaging is to detect flows relative to anatomical structures in the body, it is usually important that the F-mode image be properly registered with the B-mode image on-screen. Since this technique relies on the correlation of signal obtained from one pulse versus the subsequent pulse, and since vesicles can be destroyed by the first pulse, an F-signal is generated that is not related to motion. This loss of correlation can be shown in a variety of display formats but is typically displayed in color.

In D-mode (spectral Doppler) acquisition, the ultrasound system fires a beam and processes the return signal for a single target. Spectral Doppler information can be obtained by transmitting and receiving either continuous wave (CW) or pulsed wave (PW) ultrasonic energy. In CW Doppler acquisition, for example, Power Doppler (Doppler angiography), the ultrasound receiver continuously receives echoes from all objects within the receiver's area of sensitivity in the body, and cannot isolate information received from any specific range interval. CW Doppler is most useful where the instrument's area of sensitivity can be adjusted, either by physical placement of the probe or by beamforming, or both, to include only the desired target. n PW Doppler acquisition, the ultrasound instrument receives echoes from individual pulses, the timing of which implies a range interval within the body of the object which produced the echo. A clinician typically selects a range interval within which the target is expected to be located.

In D-mode acquisition, it is desirable to be able to produce detailed quantitative measurements over a very large range of signal levels (dynamic range). D-mode information is processed by the ultrasound system to display either the velocity spectrum of the target, plotted against time, or to provide an audio output carrying similar information. Spectral Doppler acquisition is described in Liv Hatle, M. D. & Bjorn Angelsen, Dr. Techn., “Doppler Ultrasound in Cardiology” (1st ed. 1982) and (2d ed. 1984), incorporated herein by reference in its entirety.

In addition to B-, F- and D-mode acquisition, a fourth mode also exists, known as M-mode, but this is merely a different display modality for data acquired in a manner similar to B- or F-mode acquisition. The requirements for M-mode acquisition are not significantly different from those for B- or F-mode acquisition. Alternatively, or in addition, 3-dimensional ultrasound is also contemplated, wherein 3-D scans require special probes and software to accumulate and render the images.

Additional diagnostic techniques contemplated for use in the present invention are well known in the art, and are described, for example, in Gamsu et al., Diagnostic Imaging Review (W B Saunders Co 1998), the disclosure of which are incorporated by reference herein in its entirety.

In the case of diagnostic applications (such as ultrasound, CT, MRI, and the like), energy, such as ultrasonic energy, may be applied to at least a portion of the patient to image the target tissue. A visible image of an internal region of the patient may be then obtained.

X-ray imaging: Detectably-labeled in vivo tumor diagnostics, PWM-poly IC or PWM-PHA-poly IC may comprise an X-ray detectable compound, such as bismuth (III), gold (III), lanthanum (III) or lead (II); a radioactive ion, such as copper , gallium⁶⁷, gallium⁶⁸, indium¹¹¹ indium¹¹³, iodine¹²³, iodine¹²⁵, iodine¹³¹, mercury , mercury²⁰³, rhenium¹⁸⁶, rhenium¹⁸⁸, rubidium⁹⁷, rubidium¹⁰³, technetium^{99m} or yttrium⁹⁰; a nuclear magnetic spin-resonance isotope, such as cobalt (II), copper (II), chromium (III), dysprosium (III), erbium (III), gadolinium (III), holmiuim (III), iron (II), iron (III), manganese (II), neodymium (III), nickel (II), samarium (III), terbium (III), vanadium (II) or ytterbium (III); or rhodamine or fluorescein.

These aspects of the invention are preferred for use in tumor imaging methods and combined tumor treatment and imaging methods. PWM-poly IC or PWM-PHA-poly IC compositions that are linked to one or more detectable agents are envisioned for use in imaging per se, or for pre-imaging the tumor to form a reliable image prior to treatment. Such compositions and methods can also be applied to the imaging and diagnosis of any other tumor or condition, particularly malignant and non-malignant tumors, and conditions in which an internal image is desired for diagnostic or prognostic purposes or to design treatment.

The imaging which can utilize antibodies directed to PWM or the PWM-poly IC or PWM-PHA-poly IC compositions will generally comprise an operatively attached, or conjugated to, a detectable label. Methods of making antibodies are known to one of ordinary skill in the art, including, but not limited to, in vivo immunization, in vitro immunization, and phage display methods. See, e.g., Sutcliffe et al., supra; Wilson et al., supra, and Bittle et al., J Gen. Virol., 66:2347-2354 (1985). If in vivo immunization is used, animals may be immunized with free peptide; however, anti-peptide antibody titer may be boosted by coupling the peptide to a macromolecular carrier, such as keyhole limpet hemacyanin (KLH) or tetanus toxoid. For instance, peptides containing cysteine residues may be coupled to a carrier using a linker such as maleimidobenzoyl-N-hydroxysuccinimide ester (MBS), while other peptides may be coupled to carriers using a more general linking agent such as glutaraldehyde. Animals such as rabbits, rats and mice are immunized with either free or carrier-coupled peptides, for instance, by intraperitoneal and/or intradermal injection of emulsions containing about 100 μg of peptide or carrier protein and Freund's adjuvant or any other adjuvant known for stimulating an immune response. Several booster injections may be needed, for instance, at intervals of about two weeks, to provide a useful titer of anti-peptide antibody which can be detected, for example, by ELISA assay using free peptide adsorbed to a solid surface. The titer of antibodies in serum from an immunized animal may be increased by selection of antibodies, for instance, by adsorption to the peptide on a solid support and elution of the selected antibodies according to methods well known in the art.

The antibodies of the present invention may be generated by any suitable method known in the art. The antibodies of the present invention can comprise polyclonal antibodies. Methods of preparing polyclonal antibodies are known to the skilled artisan (Harlow, et al., Antibodies: A Laboratory Manual, (Cold spring Harbor Laboratory Press, 2^{nd} ed. (1988), which is hereby incorporated herein by reference in its entirety). For example, PWM-poly IC or PWM-PHA-poly IC or PWM alone can be administered to various host animals including, but not limited to, rabbits, mice, rats, etc. to induce the production of sera containing polyclonal antibodies specific for the antigen. The administration of the polypeptides of the present invention may entail one or more injections of an immunizing agent and, if desired, an adjuvant. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. For the purposes of the invention, “immunizing agent” may be defined as a polypeptide of the invention, including fragments, variants, and/or derivatives thereof, in addition to fusions with heterologous polypeptides and other forms of the polypeptides as may be described herein.

Typically, the immunizing agent and/or adjuvant will be injected in the mammal by multiple subcutaneous or intraperitoneal injections, though they may also be given intramuscularly, and/or through IV. The immunizing agent may include polypeptides of the present invention or a fusion protein or variants thereof. Depending upon the nature of the polypeptides (i.e., percent hydrophobicity, percent hydrophilicity, stability, net charge, isoelectric point etc.), it may be useful to conjugate the immunizing agent to a protein known to be immunogenic in the mammal being immunized. Such conjugation includes either chemical conjugation by derivatizing active chemical functional groups to both the polypeptide of the present invention and the immunogenic protein such that a covalent bond is formed, or through fusion-protein based methodology, or other methods known to the skilled artisan. Examples of such immunogenic proteins include, but are not limited to keyhole limpet hemocyanin, serum albumin, bovine thyroglobulin, and soybean trypsin inhibitor. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum. Additional examples of adjuvants which may be employed includes the MPL-TDM adjuvant (monophosphoryl lipid A, synthetic trehalose dicorynomycolate). The immunization protocol may be selected by one skilled in the art without undue experimentation.

The antibodies of the present invention can also comprise monoclonal antibodies. Monoclonal antibodies may be prepared using hybridoma methods, such as those described by Kohler and Milstein, Nature, 256:495 (1975) and U.S. Pat. No. 4,376,110, by Harlow, et al., Antibodies: A Laboratory Manual, (Cold spring Harbor Laboratory Press, 2^{nd} ed. (1988), by Hammerling, et al., Monoclonal Antibodies and T-Cell Hybridomas (Elsevier, N.Y., (1981)), or other methods known to the artisan. Other examples of methods which may be employed for producing monoclonal antibodies includes, but are not limited to, the human B-cell hybridoma technique (Kosbor et al., 1983, Immunology Today 4:72; Cole et al., 1983, Proc. Natl. Acad. Sci. USA 80:2026-2030), and the EBV-hybridoma technique (Cole et al., 1985, Monoclonal Antibodies And Cancer Therapy, Alan R. Liss, Inc., pp. 77-96). Such antibodies may be of any immunoglobulin class including IgG, IgM, IgE, IgA, IgD and any subclass thereof. The hybridoma producing the mAb of this invention may be cultivated in vitro or in vivo. Production of high titers of mabs in vivo makes this the presently preferred method of production.

The antibodies or the PWM-poly IC or PWM-PHA-poly IC composition, or PWM can be labeled. “Detectable labels” are compounds or elements that can be detected due to their specific functional properties, or chemical characteristics, the use of which allows the component to which they are attached to be detected, and further quantified if desired. In antibody conjugates for in vivo diagnostic protocols or “imaging methods” labels are required that can be detected using non-invasive methods.

Many appropriate imaging agents are known in the art, as are methods for their attachment to antibodies and binding ligands (see, e.g., U.S. Pat. Nos. 5,021,236 and 4,472,509, both incorporated herein by reference). Certain attachment methods involve the use of a metal chelate complex employing, for example, an organic chelating agent such a DTPA attached to the antibody (U.S. Pat. No. 4,472,509). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

An example of detectable labels are the paramagnetic ions. In this case, suitable ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III), with gadolinium being particularly preferred.

Ions useful in other contexts, such as X-ray imaging, include but are not limited to lanthanum (III), gold (III), lead (II), and especially bismuth (III). Fluorescent labels include rhodamine, fluorescein and renographin. Rhodamine and fluorescein are often linked via an isothiocyanate intermediate.

In the case of radioactive isotopes for diagnostic applications, suitable examples include ¹⁴carbon, ⁵¹chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹¹, ⁵⁹iron, ³²phosphorus, rhenium¹⁸⁶, rhenium¹⁸⁸, ⁷⁵selenium, ³⁵sulphur, technetium^{99m} and yttrium⁹⁰. ¹²⁵I is often being preferred for use in certain embodiments, and technicium^{99m} and indiurn¹¹¹ are also often preferred due to their low energy and suitability for long range detection.

Radioactively labeled PWM-poly IC or PWM-PHA-poly IC, PWM or antibodies thereof, anti- for use in the present invention may be produced according to well-known methods in the art. For instance, intermediary functional groups that are often used to bind radioisotopic metallic ions to antibodies are diethylenetriaminepentaacetic acid (DTPA) and ethylene diaminetetracetic acid (EDTA).

Monoclonal antibodies can also be iodinated by contact with sodium or potassium iodide and a chemical oxidizing agent such as sodium hypochlorite, or an enzymatic oxidizing agent, such as lactoperoxidase. Antibodies according to the invention may be labeled with technetium-⁹⁹em by ligand exchange process, for example, by reducing pertechnate with stannous solution, chelating the reduced technetium onto a Sephadex column and applying the antibody to this column; or by direct labeling techniques, e.g., by incubating pertechnate, a reducing agent such as SNCl₂, a buffer solution such as sodium-potassium phthalate solution, and the antibody.

Any of the foregoing type of detectably labeled PWM-poly IC or PWM-PHA-poly IC, PWM or antibodies thereof, may be used in the imaging or combined imaging and treatment aspects of the present invention. They are equally suitable for use in in vitro diagnostics. Dosages for in vivo imaging embodiments are generally less than for therapy, but are also dependent upon the age and weight of a patient. One time doses should be sufficient.

The in vivo diagnostic or imaging methods generally comprise administering to a patient a diagnostically effective amount of a PWM-poly IC or PWM-PHA-poly IC, PWM or antibodies thereof, that is conjugated to a marker that is detectable by non-invasive methods. The PWM-poly IC-label conjugate or PWM-PHA-poly IC-label conjugate, PWM-label conjugate or antibody-label conjugate is allowed sufficient time to localize and bind to a tumor antigen. The patient is then exposed to a detection device to identify the detectable marker, thus forming an image of the tumor.

Chelators or bonding moieties for therapeutic radiopharmaceuticals are selected to form stable complexes with the radioisotopes that have alpha particle, beta particle, Auger or Coster-Kronig electron emissions, such as ¹⁸⁶Re, ¹⁸⁸Re, ¹⁵³Sm, ¹⁶⁶Ho, ¹⁷⁷Lu, ¹⁴⁹Pm, ⁹⁰Y, ²¹²Bi, ¹⁰³Pd, ¹⁰⁹Pd, ¹⁵⁹Gd, ¹⁴⁰La, 198Au, ¹⁹⁹Au, ¹⁶⁹Yb, ¹⁷⁵Yb, ¹⁶⁵Dy, ¹⁶⁶Dy, ⁶⁷Cu, ¹⁰⁵Rh, ¹¹¹Ag, and ¹⁹²Ir. Chelators for rhenium, copper, palladium, platinum, iridium, rhodium, silver and gold isotopes are selected from diaminedithiols, monoamine-monoamidedithiols, triamide-monothiols, monoamine-diamide-monothiols, diaminedioximes, and hydrazines. Chelators for yttrium, bismuth, and the lanthanide isotopes are selected from cyclic and acyclic polyaninocarboxylates such as DTPA, DOTA, DO3A, 2-benzyl-DOTA, alpha-(2-phenethyl)1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxymethyl)aminomethyl)-4′-(3-amino-4-methoxyp henyl)-2,2′:6′,2″-terpyridine.

Chelators for magnetic resonance imaging contrast agents are selected to form stable complexes with paramagnetic metal ions, such as Gd(III), Dy(III), Fe(III), and Mn(II), are selected from cyclic and acyclic polyaminocarboxylates such as DTPA, DOTA, DO3A, 2-benzyl-DOTA, alpha-(2-phenethyl) 1,4,7,10-tetraazacyclododecane-1-acetic-4,7,10-tris(methylacetic)acid, 2-benzyl-cyclohexyldiethylenetriaminepentaacetic acid, 2-benzyl-6-methyl-DTPA, and 6,6″-bis[N,N,N″,N″-tetra(carboxyrnethyl)aminomethyl)-4′-(3-amino-4-methoxyphenyl)-2,2′:6′,2″-terpyridine.

Suitable reducing agents for the synthesis of the radiopharmaceuticals of the present invention include stannous salts, dithionite or bisulfite salts, borohydride salts, and formamidinesulfinic acid, wherein the salts are of any pharmaceutically acceptable form. The preferred reducing agent is a stannous salt. The amount of a reducing agent used can range from 0.001 mg to 10 mg, or more preferably from 0.005 mg to 1 mg.

The indium, copper, gallium, silver, palladium, rhodium, gold, platinum, bismuth, yttrium and lanthanide radiopharmaceuticals of the present invention can be easily prepared by admixing a salt of a radionuclide and a reagent of the present invention, in an aqueous solution at temperatures from 0 to 100° C. These radionuclides are typically obtained as a dilute aqueous solution in a mineral acid, such as hydrochloric, nitric or sulfuric acid. The radionuclides are combined with from one to about one thousand equivalents of the reagents of the present invention dissolved in aqueous solution. A buffer is typically used to maintain the pH of the reaction mixture between 3 and 10.

The gadolinium, dysprosium, iron and manganese metallopharmaceuticals of the present invention can be easily prepared by admixing a salt of the paramagnetic metal ion and a reagent of the present invention, in an aqueous solution at temperatures from 0 to 100° C. These paramagnetic metal ions are typically obtained as a dilute aqueous solution in a mineral acid, such as hydrochloric, nitric or sulfuric acid. The paramagnetic metal ions are combined with from one to about one thousand equivalents of the reagents of the present invention dissolved in aqueous solution. A buffer is typically used to maintain the pH of the reaction mixture between 3 and 10.

The total time of preparation will vary depending on the identity of the metal ion, the identities and amounts of the reactants and the procedure used for the preparation. The preparations may be complete,-resulting in >80% yield of the radiopharmaceutical, in 1 minute or may require more time. If higher purity metallopharmaceuticals are needed or desired, the products can be purified by any of a number of techniques well known to those skilled in the art such as liquid chromatography, solid phase extraction, solvent extraction, dialysis or ultrafiltration.

Buffers useful in the preparation of metallopharmaceuticals useful for the preparation of said radiopharmaceuticals include but are not limited to phosphate, citrate, sulfosalicylate, and acetate. A more complete list can be found in the United States Pharmacopeia. Lyophilization aids useful in the preparation of radiopharmaceuticals include but are not limited to mannitol, lactose, sorbitol, dextran, Ficoll, and polyvinylpyrrolidine (PVP).

Stabilization aids useful in the preparation of metallopharmaceuticals and radiopharmaceuticals include but are not limited to ascorbic acid, cysteine, monothioglycerol, sodium bisulfite, sodium metabisulfite, gentisic acid, and inositol.

Solubilization aids useful in the preparation of metallopharmaceuticals and for the preparation of radiopharmaceuticals include but are not limited to ethanol, glycerin, polyethylene glycol, propylene glycol, polyoxyethylene sorbitan monooleate, sorbitan monoloeate, polysorbates, poly(oxyethylene)poly(oxypropylene)poly(oxyethylene) block copolymers (Pluronics) and lecithin. Preferred solubilizing aids are polyethylene glycol, and Pluronics.

Bacteriostats useful in the preparation of metallopharmaceuticals and radiopharmaceuticals include but are not limited to benzyl alcohol, benzalkonium chloride, chlorbutanol, and methyl, propyl or butyl paraben.

The diagnostic radiopharmaceuticals are administered by intravenous injection, usually in saline solution, at a dose of 1 to 100 mCi per 70 kg body weight, or preferably at a dose of 5 to 50 mCi. Imaging is performed using known procedures.

The magnetic resonance imaging contrast agents of the present invention may be used in a similar manner as other MRI agents as described in U.S. Pat. Nos. 5,155,215; 5,087,440; Margerstadt et al., Magn. Reson. Med., 1986, 3, 808; Runge et al., Radiology, 1988, 166, 835; and Bousquet et al., Radiology, 1988, 166, 693. Generally, sterile aqueous solutions of the contrast agents are administered to a patient intravenously in dosages ranging from 0.01 to 1.0 mmoles per kg body weight.

For use as X-ray contrast agents, the compositions of the present invention should generally have a heavy atom concentration of 1 mM to 5 M, preferably 0.1 M to 2 M. Dosages, administered by intravenous injection, will typically range from 0.5 mmol/kg to 1.5 mmol/kg, preferably 0.8 mmol/kg to 1.2 mmol/kg. Imaging is performed using known techniques, preferably X-ray computed tomography.

The ultrasound contrast agents of the present invention are administered by intravenous injection in an amount of 10 to 30 μL of the echogenic gas per kg body weight or by infusion at a rate of approximately 3 μL/kg/min. Imaging is performed using known techniques of sonography.

Administration of Imaging Agents

Any of the above described imaging or contrast agent for imaging tumors, including tumors in systems such as the lymphatic system may be administered to a vertebrate subject, such as a bird or a mammal. As employed herein, the term “lymphatic system” refers to cells, tissues or organs which comprise or are associated with the lymph system, including lymph nodes, lymph vessels, lymph canals, lymph cells, macrophages, injection situs, and the like. Preferably, the vertebrate is a human, and the lymph structure of interest, such as the lymph nodes, lymph vessels, and the like, can be imaged with any of the techniques described herein. This can be useful, e.g., for detecting the lymph nodes, tumors, necrotic regions, and infected regions.

Major areas of interest for imaging areas to assess blood supply and tissue viability, include regional spread of neoplastic and infectious lesions of the breast, colon and rectum, prostate, ovary, testes, skin cancer, and the like. Major lymph nodes involved in these various lesions include axillary and internal mammary nodes in the chest, and the pararectal, anterior pelvic (obturator), internal iliac (hypogastric), presacral, external and common iliac, and para-aortic nodes. Thus, applications where lymphographic imaging would be useful include, but are not limited to, pathological lesions affecting the major organs of the chest, abdomen and pelvis, as well as the skin, from which the regional and, subsequently, more distant lymphatics can be involved. A preferred method of imaging is MR. However, other methods are also useful. The different modes of visualization are known in the art, as well as suitable modes of administration of contrast agents, are discussed, for example, in Vogl et al. (Acta Radiol. (1997) Supp 412:47-50), the disclosure of which is hereby incorporated by reference in its entirety.

As those skilled in the art would recognize, administration of the imaging or contrast agents (agent) described herein, as well as the auxiliary materials, can be carried out in various fashions which are not intravascular, including parenteral. Parenteral administration, which is preferred, includes administration by the following routes: intramuscular, percutaneous, directly in the lymphatic vessel or the lymph node, intraepiderrnal, intramedullary, intramural or intraparenchymal, interstitially, intraperitoneal, intrathecal, subcutaneous, intrasynovial, transepithelial (including transdermal), dermal, in the tumor or pathologic process itself, and the like. Preferably, the agent is administered by interstitial injection (or other interstitial administration) in the vicinity of the tissue, organ, lymph node and the like, to be imaged, including subcutaneous (under or in the skin) and intraparenchymal (into an organ) injection, but not intraperitoneal injection (into a body cavity). In the case of cancer patients, the agent is preferably injected in proximity to the cancer. The agent can also be injected by a combination of two or more parenteral modes, for example intramuscular, subcutaneous, and in the pathologic process, insuring its accretion in the lymphatic structure of interest. Upon administration, the contrast agent is preferably taken up by the lymphatic system, generally localizing in lymph nodes (preferably the sentinel lymph node) afferent to the uptake site. Thus, preferably, the contrast agent would follow the same route as a metastatic tumor cell would be likely to follow within the lymphatic system.

Suspensions or formulations comprising agents are administered in a manner compatible with the route of administration, the dosage formulation, and in a diagnostic effective amount. It is anticipated that dosages between about 0.1 to about 30 ml of the agent (about 10 micrograms up to about 0.1 milligram per kilogram of body weight) per day will be used for diagnostic applications. In accordance with a preferred embodiment of the present invention, a small quantity of the agent (e.g., about 0.1 ml/Kg based on the body weight of the vertebrate) is introduced into the animal per injection site, but this can vary depending on the site and the number of injections. Other quantities of the agent, such as from about 0.005 ml/Kg to about 1.0 ml/Kg, are also contemplated for use in the practice of the present invention. Volumes of the agent will normally vary somewhat depending upon the site of injection, the concentration and activity of the preparation, the number of injections to be used, the particular contrast agent employed, the characteristics of the tissue desired, the degree and duration of effect desired, the judgment of the practitioner, as well as properties peculiar to each individual. Moreover, suitable dosage ranges for systemic application depend on the route of administration. Adjustment of these parameters will be conventional for the ordinary skilled clinician. Suitable regimes for initial administration are variable, but are typified by an initial administration followed by repeated doses at one or more intervals.

In addition, the agent may be in the form of a sterile injectable suspension or formulation comprising contrast agents combined with suitable carriers. Suitable carriers include non-toxic parenterally-acceptable sterile aqueous or nonaqueous solutions, suspensions, or emulsions, including the auxiliary or stabilizing materials, surfactants, and the like (each which have been described herein). This suspension may be formulated according to known methods using suitable dispersing or wetting agents and suspending agents, including the auxiliary or stabilizing materials described herein. They can also be manufactured in the form of sterile water, or some other sterile injectable medium immediately before use. Sterile, fixed oils are conventionally employed as a solvent or suspending medium. For this purpose any bland fixed oil may be employed including synthetic mono- or diglycerides, fatty acids (including oleic acid), naturally occurring vegetable oils like sesame oil, coconut oil, peanut oil, cottonseed oil, etc., or synthetic fatty vehicles like ethyl oleate, or the like. They may be sterilized, for example, by filtration through a bacteria-retaining filter, by incorporating sterilizing agents into the formulations, by irradiating the formulations, by heating the formulations, and the like. Sterile injectable suspensions may also contain adjuvants such as preserving, wetting, emulsifying, and dispersing agents. Buffers, preservatives, antioxidants, and the like can also be incorporated as required.

The imaging agent will normally be administered at a site and by means that insure that it is mobilized and taken up into the system. This will vary with the system to be imaged. Multiple injection sites may be preferable in order to permit proper drainage to the region under investigation. In some cases, injections around the circumference of a tumor or biopsy site is desired. In other cases, injection into a particular sheath or fossa is preferred. Injection into the webs of the fingers or toes is a common mode used to study, for exanple, peripheral lymphatics. The agent can be administered to the subject either pre-operatively and/or intra-operatively to localize the desired region. It is recognized that the present invention, preferably, allows immediate and real-time identification of the lymph vessel and the sentinel draining node following percutaneous injection of the agent in a region of interest. Administration of the agent does not require significant lead time to reach the region of interest. In addition, additional methodology can be employed to modify or alter the transport of the contrast agent, including massaging the injection site or stimulating flow by exercise to facilitate transport of the contrast agent to the structure of interest.

As discussed, the invention method can be used to visualize damaged tissues, lesions, tumors and the like. For example, to visualize the regions associated with genitourinary cancers or lesions, bilateral deep perianal injection of the contrast agent into the ischiorectal fossa is effective. The patient can be placed in the lithotomy position and about 1 ml of the contrast agent is introduced bilaterally into the ischiorectal fossa, e.g., with a 22 gauge needle, to a depth of about 1.5 inches just lateral to the anal margin, at the 9 and 3 o'clock positions. The patient may also lie on the side if achieving the lithotomy position is not possible. Subcutaneous dorsal pedal injection of about 0.5 ml of the contrast agent may also be made, e.g., using a 23 gauge half-inch needle in the first interdigital spaces bilaterally. In certain cases, such as testicular or prostatic cancer or some cases of rectal carcinoma, intratumoral or peritumoral injection of imaging agents can be effective.

The present method also has applicability in locating the sentinel nodes associated with breast tumor. Images of axillary, subclavian and supraclavicular nodes may be obtained by injecting the contrast agent into and around the tumor and below the skin adjacent to the tumor or the medial surface of the upper arms (ipsilateral and contralateral) of patients with breast cancer. A unilateral injection can be made in the subcostal site ipsilateral to the tumor, and then repeated later on the contralateral side to observe cross drainage between the ipsilateral and contralateral nodes. Alternatively, for example, for visualization of the internal mammary lymphatics in breast cancer, the contrast agent is injected into the posterior rectus sheath at the insertion of the diaphragm in the subcostal site, using about 1 ml of the contrast agent. By injecting a contrast agent in the vicinity of the tumor, the practitioner will know that the lymph duct involved and leading to the sentinel node will be directed toward the axillary, internal mammary, or supraclavicular chain wherein imaging is effected at appropriate times after each injection.

Another approach is to inject about 0.5 to 1 ml of contrast agent around the areola tissue of the breasts bilaterally, and then imaging the axillary, internal mammary, or supraclavicular chains. In addition to periareolar injection, interdigital administration of the contrast agent may be used for visualization of axillary lymphatics (see, DeLand et al., (1980), Cancer Res. 40:2997-3001). Combined interdigital and periareolar administration of the contrast agent can provide increased accuracy to demonstrate increased uptake in affected axillary nodes. Intratumoral injection of the contrast agent can also be performed in patients with breast cancer or melanoma and is a useful mode of administration for certain cases.

Preferably, the contrast agents persist for a sufficient amount of time following administration to allow measurements of the rate of increase in contrast agent levels in the target region and the determination of maximum signal strength. In this respect preferred imaging agents have a half life of at least about 1 minute following administration. More preferably the imaging agents have a half life of at least about 2, 3, 5, 10 or 30 minutes or more following administration. However, those skilled in the art will appreciate that the disclosed invention may be practiced by continuous administration of an imaging agent having any half life including those with half lives on the order of seconds or tens of seconds.

In accordance with yet another embodiment of the present invention, the sentinel node is then removed for evaluation as to the presence or absence of neoplastic diseases or disorders, including metastasis. Thus, the diagnostic procedure is minimally invasive, as other non-affected regional axillary nodes are not disturbed. When compared with the conventional surgical protocols of removing essentially all regional lymph nodes at the axilla, the minimally invasive aspect of the present methodology immediately becomes apparent.

Kits and Formulations

The invention also provides a kit for reducing the rate of tumor growth in a subject. The kit of the invention includes a composition comprising PWM-poly IC or PWM-PHA-poly IC and a pharmaceutically acceptable carrier as well as printed instructions for using the composition to reduce the rate of tumor growth in a subject.

Active components can be present in solid, semi-solid or liquid form. Solid forms include for example, powders, granules and flakes. Semi-solid forms include, for example, gels, creams, gelatins and ointments. These and other active agents embraced by the present invention are known to those of ordinary skill in the art and, in most cases, are commercially available from suppliers such as Compound Solutions, Inc., Escondido, Calif. Information on these and other active and inactive agents embraced by the invention, and their commercial suppliers is available from various trade manuals, most particularly, Remington's Pharmaceutical Sciences, United States Pharmacopoeia (USP), National Formulary (NF), Merck Index, Physician's Desk Reference (PDR) and Chemical Abstracts.

The kits of the invention will also generally contain at least one inactive agent. As used herein, inactive agents are agents which do not provide any therapeutic benefit to the subject to whom they are administered. Instead, inactive agents can function in many other ways such as to provide a base in which the active agent can be dissolved or suspended, to dilute the active agent in order to provide proper doses upon administration, to facilitate the dissolution or suspension of the active agent, or to prevent oxidation of the active agent by removing air bubbles from the final compounded suspension. In some embodiments of the invention, the kits lack an inactive agent, and rather contain two or more active agents.

Base agents such as creams, oils, gels or ointments are suitable for topical or suppository applications. The choice of suitable inactive base agent for use in the kits of the invention will depend upon the active agent to be compounded. Suitable base agents will be known to the ordinary artisan. Alternatively, Remington's Pharmaceutical Sciences, the Physician Desk Reference (PDR) or other manuals as listed above, can be consulted in making this determination.

Examples of inactive base agents or components include, for example, lanolin, hydrophilic ointment, white ointment, yellow ointment, polyethylene glycol ointment, petrolatum, hydrophilic petrolatum, white petrolatum, rose water ointment, squalene, hydrogenated vegetable oil (Type II), ultrasound gel, pluronic lecithin organogel (PLO) gel, cream.

The term “petrolatum” as used herein means petrolatum ointment, petrolatum gel or petrolatum cream, all of which are commercially available. It is well within the realm of the ordinary pharmaceutical artisan to determine which form of petrolatum is most appropriate for a specific kit.

A commercially available ultrasound base is either POLYSONIC™ (ultrasound gel) ultrasound lotion or Aquasonic ultrasound 100 gel manufactured by Parker Laboratories, Inc. (Fairfield, N.J.) or EcoGel 100 or EcoGel 200 manufactured by Eco-Med (Mississauga, Ontario, Canada), the compositions of which may include cetyl alcohol, liquid paraffin, polymer, surfactants, preservatives such as propyl paraben and methyl paraben in bacteriostatic concentration, fragrance, and reverse osmosis water. As used herein, a gel is a base with a higher viscosity than a lotion. The physical characteristics of the POLYSONIC™ (ultrasound gel) ultrasound lotion and the EcoGel 100 include pH range of 6.5-7.0, density of 1.04 g/cm³, viscosity of 35,000 to 70,000 cps and acoustic impedence of 1.60 (10⁵ g/cm² sec). The physical characteristics of Aquasonic ultrasound 100 gel or EcoGel 200 are similar to those of POLYSONIC™ (ultrasound gel) ultrasound lotion and EcoGel 100 except that their viscosity is 80,000 to 110,000 cps. These lotions and gels are available in a clear, colorless form or in a blue colored form.

Liquid bases are recommended for orally administered pharmaceuticals. In some embodiments of the invention, at least one active agent, e.g. PWM-poly IC-PHA, will be supplied already co-mingled with an inactive agent. Examples of this include the combination of magnesium hydroxide and aluminum hydroxide (commercially available as MAALOX™ (magnesium hydroxide/aluminum hydroxide)), and diphenhydramine HCl (commercially available as BENADRYL™ (diphenhydramine hydrochloride)). Both MAALOX™ (magnesium hydroxide/aluminum hydroxide) and BENADRYL™ (diphenhydramine hydrochloride) are supplied by their respective manufacturers as a combination of active and inactive agents.

Sterile base solutions are preferred for parenteral (i.e., injection), aerosol (i.e., inhalation) and ophthalmic routes of administration. The administration may, for example, be intravenous, intraperitoneal, intramuscular, intracavity, subcutaneous or transdermal. Preparations for parenteral administration includes sterile aqueous or nonaqueous solutions, suspensions and emulsions. The compounded pharmaceuticals, preferably those intended for parenteral, inhalation or ophthalmic routes of administration, may be prepared and administered in inactive agents which are pharmaceutically-acceptable. As used herein, a pharmaceutically-acceptable carrier means a non-toxic material that does not interfere with the effectiveness of the biological activity of the active agents and that is compatible with the biological systems such of a tissue or organism. The physiologically acceptable carrier must be sterile for in vivo administration. Pharmaceutically acceptable carriers include diluents, fillers, salts, buffers, stabilizers, solubilizers and other materials which are well-known in the art. The characteristics of the carrier will depend on the route of administration. In general, pharmaceutically-acceptable agents or carriers are well-known to those of ordinary skill in the art. In some embodiments, suitable sterile solutions include albuterol and ipratropium inhalation solution; papaverine, phentolamine and prostaglandin injection solution; fentanyl citrate injection solution and cyclosporine ophthalmic drops.

Examples of nonaqueous solvents are propylene glycol, polyethylene glycol, vegetable oil such as olive oil, an injectable organic esters such as ethyloliate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers, (such as those based on Ringer's dextrose), and the-like. Preservatives and other additives may also be present such as, for example, antimicrobials, antioxidants, chelating agents, and inert gases and the like. Those of skill in the art can readily determine the various parameters for preparing these alternative pharmaceutical compositions without resort to undue experimentation.

Inactive agents may also include components which function to preserve the integrity of the compounded formulation. This latter category of inactive agents includes, for example, anti-foaming agents. Anti-foaming agents are agents which function to remove unwanted air trapped in a composition, perhaps during mixing or agitation. The use of anti-foaming components is particularly useful in the preparation of pharmaceuticals to be used for ultrasound imaging due to the impedance of signal transmission by air bubbles. Examples of other anti-foaming agents useful in the compositions of the invention include bisphenylhexamethicone, dimethicone, dimethiconol, hexamethyldisiloxane, hexyl alcohol, isopropyl alcohol, petroleum distillates, phenethyl disiloxane, phenyl trimethicone, polysilicone-7, propyl alcohol, silica dimethyl silylate, silica silylate, tetramethyl decynediol and trimethylsiloxysilicate. A preferred anti-foaming agent is simethicone. Simethicone is a mixture of about 90% dimethicone and 10% silicone dioxide (w/w). Simethicone is used extensively as an anti-gas agent in pharmaceutical products such as GAS-Xm (simethicone), MAALOX™ (magnesium hydroxide/aluminum hydroxide), MYLANTA™ (aluminum, magnesium simethicone), PHAZYME™ (simethicone), GENAZYME™ (simethicone), and MYLICON™ (simethicone) Drops. Simethicone may be used as an anti-foaming agent in any of the formulations embraced by the invention.

Other inactive agents which can be included in the formulations of the invention include stabilizers such as citric acid, anti-oxidants such as sodium metabisulfite and preservatives such as methyl or propyl paraben.

Another class of inactive agents is suspending agents. Suspending agents are agents which facilitate the suspension and in some cases the dissolution of an active agent in a base. Generally, suspending agents ensure more uniform mixing of active and base components. In order to administer a more uniform dose of a compounded pharmaceutical to a patient, the compounded components must be properly and homogeneously combined. If the active agent is present as a powder, a uniform dispersion is sometimes difficult to achieve using the traditional form of compounding.

A subcategory of suspending agents are solubilizers. Solubilizers are agents which facilitate the dissolution of a solid or, in some cases, a semi-solid agent in a base inactive agent. In some embodiments of the invention, a solid-form active agent may be dissolved in a suspending agent, prior to mixing it with the base agent. Conversely, the suspending agent and the base agent may be prepackaged together, particularly if the concern is ensuring the uniform blending of active agent within the base component rather than the loss of solid (i.e., powdery) active agent. In still other variations, the suspending agent may be premixed with the base inactive agent.

Suitable suspending agents useful in the compositions of the invention include, but are not limited to, glycerin, hexylene glycol, propylene glycol, sorbitol, acacia, cholesterol, diethanolamine (adjunct), glyceryl monostearate, lanolin alcohols, lecithin, mono- and di-glycerides, monoethanolamine (adjunct), oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, propylene glycol diacetate, propylene glycol monostearate, sodium lauryl sulfate, sodium stearate, sorbitan monolaurate, sorbitan monooleate, sorbitan monopalmitate, sorbitan monstearate, stearic acid, trolamine, emulsifying wax, benzalkonium chloride, benzethonium chloride, cetylpyridinium chloride, docusate sodium, nonoxynol 9, nonoxynol 10, octoxynol 9, polyoxyl 50 stearate, and tyloxapol.

Still other suspending agents include humectants and wetting agents. Humectants are agents which retain moisture. Examples of humectants include but are not limited to glycerin, hexylene glycol, propylene glycol and sorbitol. The amounts of base and non-base inactive agents will also depend upon the particular compounded pharmaceutical to be made. Base agents can be provided in quantities corresponding to final compounded preparations which contain 0.5% to 99.99% of base agent, either in weight or in volume. In preferred embodiments, the final concentration of the base agent is 20%-80%. In even more preferred embodiments, the final concentration of the base agent is 40%-80%.

Generally, the amounts of non-base agents will be sufficient to provide final formulations in which each non-base inactive agent represents 0.01%-50% (w/w) of the composition. Suspending agents may represent 1%-50% (w/w) of the final formulation. Preferably, suspending agents will represent 1%-40% and even more preferably, they will represent 5%-30% of the final formulation. Anti-foaming agents may represent 0.01% to 20% (w/w) of the final formulation. More preferably, anti-foaming agents represent 0.05% to 10% of the final formulation and even more preferably, they represent 0.1% to 5% of the final formulation.

In some preferred embodiments, the single or multiple unit of use kits are designed to yield, after the physical mixing of active and inactive agents, compounded pharmaceutical formulations comprising 1%, 5%, 10% to 99% w/w of PWM-poly IC or PWM-PHA-poly IC.

The kits of the invention will provide each and every component required for preparing a given compounded pharmaceutical in pre-measured quantities. The measuring of each component will be performed using current Good Manufacturing Practices (cGMP, as legislated by the Code of Federal Regulations or CFR), as will the packaging and labeling of each component and the final packaging and labeling of the kit in its entirety. In this way, the kits are standardized and variations from batch to batch will be minimal or non-existent and the precision and accuracy in the measurement of individual components will be improved considerably over the methods currently used by pharmacists. Instructions may be provided as separate from any container, but still contained in the kit. Alternatively, instructions may be located on a container, for example, on an exterior surface or on an interior surface such as a lid.

Both the active and the inactive agents of the kit are provided in containers. Since the kit will contain at least one active and at least one inactive agent, or at least two active agents pre-formulated with inactive agents, the minimum number of containers in a given kit will be two. In preferred embodiments, the maximum number of containers in a kit will be less than or equal to four. The containers may be formed in any size or shape useful for the mixing or transferring of components from one container to another. For example, each container may be in the form of vials, bottles, squeeze bottles, jars, sealed sleeves, envelopes. or pouches, tubes or blister packages or any other suitable form provided the container is sealed so as to prevent premature mixing of components. As used herein, a container may also be a compartment or a chamber within a vial, a tube, ajar, or an envelope, or a sleeve, or a blister package or a bottle, provided that the contents of one compartment are not able to associate physically with the contents of another compartment prior to their deliberate mixing by a pharmacist or physician.

The invention intends to provide within a single kit all the necessary components, containers and stirring or mixing elements for preparing a unit of use compounded pharmaceutical without the need for other accessories. The kits of the invention may also contain items such as gloves or spill pads. Individuals skilled in the art can readily modify the choice of container to suit the individual components housed and mixed therein.

In some embodiments of the invention, the final compounded formulation will be provided to the patient in the container originally housing the inactive, or base, compound. In other embodiments, the final compounded formulation will be provided in the container originally housing the active agent. In still other embodiments, all the necessary components for preparing a compounded pharmaceutical are included in one container but are physically separated within such a container. For example, an inactive agent may be contained in the lower part of a container, such as a jar, and may be covered by a plastic, peel-off wrap. The active agent may be housed in this same jar, but secured to the lid of the jar and provided in a pouch or a sleeve. The ability to provide all components together in the smallest packaging arrangement may be preferable in some circumstances. Mixing elements required in the preparation of the compounded pharmaceutical may also be located within the same container, for example, secured to the inside surface of the lid of the container.

In still another embodiment of the invention, active and inactive agents are provided in adjacent compartments of a single housing container, and are mechanically removed from these compartments and into a third compartment. As an example, all the chemical components necessary to prepare a particular compounded pharmaceutical can be present in a single tube, for example, a tube similar to a toothpaste tube having an interior which is divided into separate compartments. Each of these compartments in turn house a base agent or an active agent. Either the base agent or the active agent may be premixed with an anti-foaming agent and/or a suspending agent, as described herein. By applying pressure on the tube as a whole, the components are made to exit their respective compartments. They can then be mixed either in an adjacent or a physically separate compartment. Squeezing or pressing of the outside surface of the tube may be all that is necessary to retrieve the individual components housed within the tube. In yet another embodiment, the contents of both chambers of a container can be pumped out and into a third container. In a related embodiment, it is also envisioned that rather than requiring the contents of each compartment to exit and flow into a third compartment, the components may be separated by a removable sheet or film. Thus, upon removal of such a sheet or film, the contents of the two compartments are in contact and may require only agitation or end-over-end inversion to become completely mixed. This latter embodiment would eliminate the need for a mixing element, and potentially for an exterior package particularly if the instructions are written on the container itself.

According to some aspects of the invention, each container may contain one or more active agents or one or more inactive agents. For example, in some embodiments of the invention, none of the containers may contain both an active and an inactive agent prior to mixing by the pharmacist or physician. However, the invention also provides for kits in which a container may contain an active and at least one inactive agent, such as a base agent, a suspending agent or an anti-foaming agent.

In a preferred embodiment, the active agent is provided premixed with an inactive agent. This applies mainly when PWM-poly IC or PWM-PHA-poly IC will be commercially available as a solid, for example a powder, and the pre-mixing of the powder with a suspending agent facilitates the compounding by the pharmacist or physician. In yet other embodiments, at least two of the inactive agents may be pre-mixed as provided in the kits of the invention.

In some embodiments, where the active agent is added to the base component, it may be desirable to provide the base component in a container which is only partially full. In preferred embodiments, the container in which the base component is situated is less than 100% full by volume. In other embodiments, the containers are 95%, 90%, 80%, 75%, 70%, 60%, 50%, 40%, 30%, 25%, 20% or less than 20% full by volume. In other embodiments, the active or inactive agents comprise a volume of their respective containers ranging from 100% to greater than 1%, and every integer there between. In preferred embodiments, the inactive agent occupies a volume of the second container which is less than or equal to the volume of the second container minus the volume of the active agent.

As used according to the invention, the active and inactive agents are physically combined by a pharmacist to produce a compounded pharmaceutical. The components of the kit can be combined by gentle agitation, shaking, stirring, folding or end-over-end inversion of the first or second container. In some instances, the proper mixing of the active and inactive agents may be accomplished simply by adding one to the other, followed by sealing and agitation of the container. This is especially the case if the components are both liquids or both semi-solids. In other instances, it may be necessary to stir the components together with a mixing element. Mixing elements are well known to a person of ordinary skill in the pharmaceutical arts and may include for example, centrifuges, a mixing rod such as a glass rod, a spoon, a spatula or a dipstick. Where required, the mixing element is provided in the kit. The presence of a mixing element will vary depending on the compounded pharmaceutical formulation to be made with the components of a kit.

The final compounded pharmaceutical may be formulated into preparations in solid. semi-solid, liquid or gaseous forms such as tablets, capsules, powders, granules, ointments, solutions, suppositories, inhalants and injections, and usual ways for oral, parenteral or surgical administration. The invention also embraces locally administering the compounded pharmaceuticals of the invention such as, for example, as implants. These formulations may be intended for oral, topical, mucosal, parenteral (e.g., injectable), rectal or vaginal administration. In preferred embodiments, the final compounded formulations may be self-administered.

The kits of the invention may also contain a package which may be compartmentalized to receive in close confinement two or more containers of the invention. In some embodiments, the package may be box-like, being made of a moderately rigid material such as cardboard or reinforced paper. In other embodiments, the package may be a bag. In still other embodiments, as described herein, there is no external packaging and all containers may be incorporated into one of the containers housing either an active or an inactive agent. This latter embodiment can be accomplished by securing containers such as pouches, sleeves or sacs, containing either active or inactive agents, as well as any mixing elements required for the compounding, to the interior of the lid of the main container. An individual skilled in the art can readily modify the package to suit the individual needs of each kit and each use. The kits of the invention further contain instructions for the proper use of the components found therein.

The kits of the invention are intended for use in the treatment or prevention of a number of disorders in a variety of subjects including humans, dogs, cats, horses, fish, pigs, cows, sheep, deer, zoo animals and laboratory animals (e.g., mice, rats, rabbits, monkeys, etc.). The invention intends to embrace unit of use kits containing the above preparations.

The following examples are offered by way of illustration, not by way of limitation. While specific examples have been provided, the above description is illustrative and not restrictive. Any one or more of the features of the previously described embodiments can be combined in any manner with one or more features of any other embodiments in the present invention. Furthermore, many variations of the invention will become apparent to those skilled in the art upon review of the specification. The scope of the invention should, therefore, be determined not with reference to the above description, but instead should be determined with reference to the appended claims along with their full scope of equivalents.

All publications and patent documents cited in this application are incorporated by reference in pertinent part for all purposes to the same extent as if each individual publication or patent document were so individually denoted. By their citation of various references in this document, Applicants do not admit any particular reference is “prior art” to their invention.

### EXAMPLES

The present invention is further illustrated by the following specific examples. The examples are provided for illustration only and are not to be construed as limiting the scope or content of the invention in any way.

### Example 1

### PWM-Poly IC Composition

Even though the initial production of IgG was low with PEG, eventually isoclass switching occurs and IgG production rises again. Ultimately we were able to abolish switching by using a small nucleotide of our own design. The engineered PWM plus the nucleotide was patented under the name Macrimmune™. Macrimmune™ consists of three major components given by injection: PEG modified PWM; Interleukin II; a cationic-lipid DNA complex;

The composition, herein termed Nutrimmune™ comprises:

| | |
|---|---|
| 1. Pokeweed mitogen (PWN) | 1 mg. |
| 2. Poly (I:C) | 1 mg, and optionally, |
| 3. Phytohemmagglutinin (PHA) | 0.5 mg |

The Poly(I:C) is enclosed in a cationic liposome of 1:1 molar ratio of DOTAP-cholesterol. DOTAP=1,2 dioleol-3-trimethylammonium-propoane. Lipid/RNA ratio set at 32 nmol lipid to 1.0 microgram RNA, or equivalents thereof.

Twenty patients were treated using the PWN-poly IC compositions. Fifteen patients went into remission after undergoing treatment. Five patients did not follow-up and continue with the regimen. Cancers treated were breast, melanoma and lung cancer.

### Example 2

### Therapeutic Compositions

Helix pomatia lectin, is modified using 2,4-dinitrophenol (DNP). Modification is by coupling of DNP to the soluble protein. (Inman, J. K. et al., Immunochemistry, 10:165, 1973). A solution of 300 mg of Helix pomatia lectin in 4.0 ml of 0.25 M potassium borate was cooled to 0° C. Fifty seven micro-moles (28.6 mg of N-(2,4-dinitrophenyl-β-alanylglycylglycine Boc hydrazide (compound “J”) were converted to the corresponding acyl azide by reaction with nitrous acid in cold dimethylformamide after removal of the Boc group.

After addition of sulfamic acid to destroy nitrous acid, the azide solution was added to the cold buffered Helix pomatia. The mixture was stirred at 0° C. for 2 days and dialyzed in the cold for 2 days against 0.1 M NaCl and 2 days against water. Outside solutions were saturated with toluene. The dialysate was lyophilized.

The resulting Helix pomatia lectin has about 10 DNP molecules attached to primarily lysine residues in the lectin molecule. This hapten conjugate is added to compound A. Compound A comprises isolated pokeweed mitogen which is coated with polyethylene glycol as described in U.S. patent application Ser. No. 09/983,129 which is incorporated herein by reference in its entirety. Compound A optionally comprises recombinant Interleukin 2 (rIL-2) of about 100,000 U/kg.

The resultant composition comprises polyethylene glycol coated pokeweed mitogen (PWM-PEG) of 10 μg PWM protein/kg; Helix pomatia lectin coupled to hapten DNP (HPL-DNP) of 30 μg/HPL protein/kg and recombinant Interleukin 2 (rIL-2) of 100,000 U/kg.

Compound B comprises a DNA nucleotide: GACGTCGACGTTAACGTCAACGTT (SEQ ID NO: 1); DOTAP (1,2dioleoyl-3-trimethylammonium-propane) and cholesterol.

Compound B is prepared as follows. DNA (SEQ ID NO: 1) is added to cationic lipids DOTAP and cholesterol in a 1:1 molar ratio. The DNA is added at a ratio of 30 nmol lipid to 1 μg DNA to a final concentration of 100 μg DNA per 0.1 ml dextrose. The dose of DNA used for immunostimulation is about 50-100 μg/kg.

The resulting composition is herein termed BiOmune.

### Example 3

### Treatment of Cancer

Protocol for treatment of breast cancer: Patient groups include women with breast cancer who have recurrent local disease or primary local disease. These women must have either not undergone chemotherapy, or are at least six weeks post chemotherapy. These women will be given systemic immune stimulation, and a breast poultice in the form of a cream. The cream will contain the Helix Lectin or a placebo. The study will be double blinded, with the stipulation that the study will be unblinded if one group shows an early advantage over the other.

Workup of the patients will consist of History and Physical exams, routine labs, color photographs of the affected breast(s). Initial NMR, PET scans and thermography will be employed to evaluate the primary and the distant lesions. The patients will be given a six-week course of therapy. On the completion of therapy, the initial scans will be repeated. The patients will then be graded as to response.

Cost: A six-week course of therapy will cost about one thousand dollars per patient for material to make the composition. The cost of material for the injectable or the oral version of composition would be about $2000/patient. The cost for medical tests and supervision is estimated at $ 6,000 per patient. If we ran 100 patients through this protocol, we would need close to one million dollars. This is a very small amount for a treatment, which we believe, will revolutionize the treatment of breast cancer.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

All references mentioned herein, are incorporated by reference in pertinent part.

What is claimed is:

1. A pharmaceutical composition comprising:
pokeweed mitogen; and,
poly(I:C).

2. The pharmaceutical composition of claim 1, wherein the pokeweed mitogen and poly(I:C) are in a ratio of 1:1 v/v respectively.

3. The pharmaceutical composition of claim 1, wherein the composition optionally comprises phytohemaglutinin.

4. The pharmaceutical composition of claim 1, wherein the composition comprises pokeweed mitogen (PWN): phytohemmagglutinin (PHA): poly(I:C) in a ratio of 1:0.5:1 v/v respectively.

5. The pharmaceutical composition of claim 1, wherein the composition further comprises recombinant Interleukin 2 (rIL-2) of about 100,000 U/kg.

6. The pharmaceutical composition of claim 1, wherein the pokeweed mitogen is PEG-modified.

7. A method of treating a cancer patient, comprising;
administering to a patient in need thereof, a composition comprising pokeweed mitogen; phytohemmaglutinin; and, poly(I:C);
contacting a tumor cell with the composition;
thereby treating the cancer patient.

8. The method of claim 7, wherein the composition comprises about 1% up to 99% w/w of pokeweed mitogen; phytohemmaglutinin; and, poly(I:C) in a ratio of 1:0.5:1 v/v respectively.

9. The method of claim 7, wherein the composition comprises about 1% to about 20% w/w of pokeweed mitogen; phytohemmaglutinin; and, poly(I:C) in a ratio of 1:0.5:1 v/v respectively.

10. The method of claim 7, wherein the composition comprising the pokeweed mitogen; phytohemmaglutinin; and, poly(I:C) in a ratio of 1:0.5:1 v/v respectively is formulated as a topical cream.

11. The method of claim 7, wherein a therapeutic effective amount of the pokeweed mitogen; phytohemmaglutinin; and, poly(I:C) composition is administered with one or more chemotherapeutic agents.

12. The method of claim 7, wherein the chemotherapeutic agent can be co-administered, precede, or administered after the composition comprising a therapeutic effective amount of the pokeweed mitogen, phytohemmaglutinin, poly(I:C) composition.

13. The method of claim 7, wherein the chemotherapeutic agent is selected from the group consisting of cyclophosphamide (CTX, 25 mg/kg/day, p. o.), taxanes (paclitaxel or docetaxel), busulfan, cisplatin, cyclophosphamide, methotrexate, daunorubicin, doxorubicin, melphalan, cladribine, vincristine, vinblastine, and chlorambucil.

14. The method of claim 7, wherein treatment results in inhibition of tumor cell growth.

15. A method of tumor imaging using X-ray, single-photon-emission tomography(SPECT), magnetic resonance imaging (MRI), or positron emission tomography (PET), comprising administering a pharmaceutically acceptable composition containing detectably labeled PWM-poly IC-, or a detectably labeled PWM molecule or an anti-PWM antibody.

16. The method of claim 7, wherein the detectable label is selected from the group consisting of: copper⁶⁷, gallium⁶⁷, gallium⁶⁸, indium¹¹¹, indium¹¹³, iodine¹²³, iodine¹²⁵, iodine¹³¹, mercury¹⁹⁷, mercury²⁰³, rhenium¹⁸⁶, rhenium¹⁸⁸, rubidium⁹⁷, rubidium¹⁰³, technetium^{99m} and yttrium⁹⁰.

17. The method of claim 7, wherein the detectable label is selected from the group consisting of: cobalt (II), copper (II), chromium (III), dysprosium (III), erbium (III), gadolinium (III), holmium (III), iron (II), iron (III), manganese (II), neodymium (III), nickel (II), samarium (III), terbium (III), vanadium (H), ytterbium (III); rhodamine or fluorescein.

18. The method of claim 7, wherein the detectable label is selected from the group consisting of: chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III), or erbium (III).

19. A kit comprising:
pokeweed mitogen;
phytohemmaglutinin; and,
poly(I:C).
